(19) United States
(12) Patent Application Publication
(10) Pub. No.: US 2025/0241950 A1
(43) Pub. Date: Jul. 31, 2025

(54) CHIMERIC ANTIGEN RECEPTOR MACROPHAGE COMPOSITIONS AND USES THEREOF

(71) Applicant: Washington University, St. Louis, MO (US)

(72) Inventors: Carl DeSelm, St. Louis, MO (US); Jin-Moo Lee, St. Louis, MO (US); Abhinav Diwan, St. Louis, MO (US)

(21) Appl. No.: 19/040,397

(22) Filed: Jan. 29, 2025

Related U.S. Application Data
(60) Provisional application No. 63/626,851, filed on Jan. 30, 2024.

(51) Int. Cl. A61K 35/15; A61K 40/17; A61K 40/31; A61P 25/28; C07K 14/535; C07K 14/735; C07K 16/18; C12N 15/86

(52) U.S. Cl. CPC A61K 35/15; A61K 40/17; A61K 40/31; A61P 25/28; C07K 14/535; C07K 14/70535; C07K 16/18; C12N 15/86; A61K 2239/13; A61K 2239/21; A61K 2239/22; C07K 2317/56; C07K 2317/622; C12N 2740/13043; C12N 2740/15043

(57) ABSTRACT

Provided are chimeric antigen receptor (CAR) that bind to beta amyloid, macrophages (CAR-Ms) that express the CAR, and compositions comprising the same. Also provided are methods for reducing one or more symptoms associated with Alzheimer's disease using the CAR-Ms.

### FEDERALLY SPONSORED RESEARCH OR DEVELOPMENT

This invention was made with Government support under Grant Number OD026427 awarded by National Institutes of Health. The Government has certain rights in this invention.

### INCORPORATION OF SEQUENCE LISTING

The present application contains a Sequence Listing which has been submitted in .XML format via Patent Center and is hereby incorporated by reference in its entirety. Said WIPO Sequence Listing was created on Jan. 29, 2025, XML copy is named 047563_797095.xml and is 25 kilobytes in size.

### FIELD OF THE INVENTION

The present disclosure relates to Chimeric antigen receptor macrophages (CAR-Ms) and use of the CAR-Ms for reducing amyloid plaque load and treating Alzheimer's disease.

### BACKGROUND OF THE INVENTION

Recent breakthroughs in liquid cancer treatment brought on by the advent of chimeric antigen receptor (CAR) T-cells inspire considerations that CAR cellular therapy, in which a specialized immune cell is modified with a CAR to both target a specific antigen and induce a desired cellular function in response to that antigen, may have broader roles in medicine beyond cancer. Indeed, the idea of a living drug that can respond in more sophisticated ways than a single small molecule is appealing for diseases that have thus far evaded conventional approaches. Beyond cancer, Alzheimer's disease (AD) is one such disease that is both growing in prevalence and treatable but not curable, despite a large number of mouse and human trials with small molecules, antibodies, and targeted therapies.

Beta amyloid (Aβ) plaque deposition is hypothesized to be a key initial trigger for Alzheimer's disease (AD) pathophysiology. Antibodies targeting Aβ have recently been shown to reduce amyloid plaque load and mitigate cognitive decline in AD patients, but have dose-limiting side effects such as amyloid-related imaging abnormalities (ARIA). Chimeric antigen receptor macrophages (CAR-Ms) have been reported to phagocytose tumor cells in an antigen-specific manner and are currently in a cancer clinical trial (NCT04660929); their use has not yet been extended to non-cancerous conditions, such as Alzheimer's disease.

Substantial evidence suggests a role for immunotherapy in treating Alzheimer's disease (AD). While the precise pathophysiology of AD is incompletely understood, clinical trials of antibodies targeting aggregated forms of beta amyloid (Aβ) have shown that reducing amyloid plaques can mitigate cognitive decline in early-stage AD patients. Therefore, there is an unmet need to develop CARs for reducing amyloid plaque load and treating Alzheimer's disease.

### SUMMARY OF THE INVENTION

Provided herein is a chimeric antigen receptor (CAR) comprising an antigen binding domain that specifically binds to beta amyloid (Aβ). The Aβ may be an aggregated form of Aβ. The antigen binding domain of the CAR may comprise an anti-Aβ antibody or an antigen binding fragment thereof. The antigen binding domain may comprise an anti-Aβ single chain variable fragment (scFv).

In one instance, the antigen binding domain comprises: (a) a heavy chain variable region comprising the amino acid sequence set forth in SEQ ID NO: 1, and a light chain variable region comprising the amino acid sequence set forth in SEQ ID NO: 2; or (b) a heavy chain variable region comprising the amino acid sequence set forth in SEQ ID NO: 15, and a light chain variable region comprising the amino acid sequence set forth in SEQ ID NO: 16.

The CAR may further comprise a linker, a transmembrane domain, and an intracellular domain. The linker may comprise the amino acid sequence set forth in SEQ ID NO: 4. The transmembrane domain may comprise the amino acid sequence set forth in SEQ ID NO: 5 or SEQ ID NO: 7. The intracellular domain may comprise a FcRγ signaling domain. The FcRγ signaling domain may comprise the amino acid sequence set forth in SEQ ID NO: 6 or SEQ ID NO: 8.

Further described herein is a vector comprising a nucleic acid encoding the CAR. The vector may be a retrovirus or a lentivirus. The retrovirus may be Moloney Murine Leukemia Virus (MuLV).

A cell expressing the CAR is further provided. The cell may be a macrophage. The cell may further express a cytokine. The cytokine may be macrophage colony-stimulating factor (M-CSF) or Granulocyte-macrophage colony-stimulating factor (GM-CSF). In one instance, the cytokine may be M-CSF and may comprise the amino acid sequence set forth in SEQ ID NO: 13 or SEQ ID NO: 14. Alternatively, the cytokine may be GM-CSF and may comprise the amino acid sequence set forth in SEQ ID NO: 17. The cytokine may be encoded by (a) the same vector encoding the CAR; or (b) a second vector.

The disclosure further encompasses a composition comprising the cell and a pharmaceutically acceptable excipient.

A method of reducing Aβ plaques in a subject in need thereof, comprising administering to the subject the cell, is further described herein. Additionally, a method of treating Alzheimer's disease in a subject in need thereof, comprising administering to the subject the cell is provided. A method of reducing one or more symptoms associated with Alzheimer's disease in a subject in need thereof, comprising administering to the subject the cell is also provided. The symptoms may comprise diffuse or compact Aβ plaques in the subject. The cell may be administered by intracranial injection. The method may induce resorption and/or degradation of Aβ plaques. The cell may further express a cytokine. The cytokine may be M-CSF or GM-CSF.

Described herein is a chimeric antigen receptor macrophage (CAR-M) comprising the amino acid sequence set forth in any one of SEQ ID NO: 11, SEQ ID NO: 12, SEQ ID NO: 20, or SEQ ID NO: 22.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1D show the generation, validation, and phenotyping CAR HoxB8 cells. FIG. 1A shows the amino acid sequence of the aducanumab based scFv and rest of the Aβ CAR construct. Aducanumab vH shows the amino acid residues of SEQ ID NO: 1, Aducanumab vL shows the amino acid residues of SEQ ID NO: 2, CAR extracellular linker, transmembrane, and intracellular domain derived from the common gamma chain (SEQ ID NO: 3) shows the amino acid residues of the extracellular linker (residues 1-5 of SEQ ID NO: 3), transmembrane domain (residues 6-26 of SEQ ID NO: 3), and intracellular domain (residues 27-68 of SEQ ID NO: 3), respectively. FIG. 1B is series of representative FACS plots showing surface expression of the Aβ CAR on untransduced, retrovirally transduced, and transduced and sorted HoxB8 cells used for downstream experiments. Numbers represent percentage of cells in the indicated gate. Representative of n>3 independent experiments. FIG. 1C is series of representative FACS plots showing surface expression of F4/80 (y-axis) and control or Aβ CAR (x-axis) on sorted HoxB8 cells differentiated into mature macrophages with M-CSF for 6 days. Representative of n>3 independent experiments. FIG. 1D is series of scatter plots showing gating strategy used for flow cytometry phenotyping of untransduced macrophages, control CAR-Ms, or Aβ CAR-Ms incubated on brain slices from aged APP/PS1 mice. Phenotyping markers were assessed on single, live, f4/80+ cells. SSC-A, side scatter area; FSC-A, forward scatter area; FSC-H, forward scatter height.

FIG. 2A-2F show the generation, validation, and phenotype of an aducanumab-based Aβ CAR-M. FIG. 2A is a schematic diagram of Aβ CAR and Control CAR constructs. scFv, single-chain variable fragment; E CAR, EphA2 scFv CAR; E(t) CAR, EphA2 truncated CAR. FIG. 2B is series of representative FACS plots of (left) F4/80 and CD64 surface expression on HoxB8 cells at day 0 and day 6 of differentiation with M-CSF; (right) surface CAR expression on cells at day 6 of differentiation. FIG. 2C is series of line plots showing in vitro uptake of AlexaFlour488 fluorescent tagged Aβ (1-42) by untransduced, control, or Aβ CAR-Ms after 2 or 4 hours of co-incubation, depicted as % uptake (left) or geometric mean fluorescence intensity (MFI, right). FIG. 2D is series of representative immunofluorescence microscopy images of Aβ CAR-Ms incubated with AF-488 tagged Aβ (1-42) for 4 hours and stained with Lamp1. FIG. 2E is a schematic of flow-based phenotyping of untransduced macrophages, control CAR-Ms, or Aβ CAR-Ms co-incubated with amyloid-laden brain slices from aged APP/PS1 mice for 44 hours prior to flow cytometry analysis. FIG. 2F is series of bar charts showing percent change in the stated marker after co-incubation of untransduced, control, or Aβ CAR-Ms on amyloid-laden brain slices from APP/PS1 mice from the expression of the same markers on the same cells incubated without brain slices. Data is represented as mean±s.e.m. from n=2 independent experiments with 3-5 technical replicates for each condition (FIG. 2C) and 5 technical replicates (FIG. 2F). Statistical significance was calculated with a repeated measures 2-way ANOVA (2c) or one-way ANOVA (FIG. 2F) with Tukey's multiple comparisons tests. For FIG. 2F, *P<0.05, **P<0.01, ***P<0.001, ****P<0.0001. ns, not significant.

FIG. 3A-3B show that an anti-AP CAR allows macrophages to take up various forms of Aβ more effectively than control CAR or untransduced macrophages. FIG. 3A is series of representative flow cytometry plots showing uptake of AF488-labeled Aβ in the indicated forms by untransduced, control CAR, or Aβ CAR macrophages after 2 hours of co-culture in vitro (y-axis). Left plot indicates background levels of AF488 fluorescence in a sample that contained macrophages only without AF488-labeled Aβ. FSC-A, forward scatter area. FIG. 3B is series of line graphs showing quantification of monomeric, oligomeric, or fibril Aβ uptake after the given amount of time, shown as AF488 mean fluorescence intensity (top), the proportion of macrophages that were AF488+(middle), or the cell count of AF488+ macrophages (bottom). Data are shown as mean±s.e.m. and is representative of 2 independent experiments with 3 technical replicates per condition per experiment. Statistical significance calculated with 2-way ANOVA with Tukey's multiple comparisons test. *P<0.05, **P<0.01, ***P<0.001, ****P<0.0001.

FIG. 4A-4G show that Aβ CAR-Ms resorb amyloid plaques of various sizes on brain slices from aged APP/PS1 mice ex vivo and degrade amyloid more efficiently than control macrophages. FIG. 4A is a schematic of ex vivo assessment of amyloid plaque phagocytosis. Adjacent brain slices from aged APP/PS1 mice were co-incubated with Aβ CAR-M or control CAR-Ms for 44 hours and plaque load was assessed with HJ3.4 or X-34 immunostaining. FIG. 4B is a series of bar graph showing assessment of plaque load. FIG. 4C is series of images showing assessment of plaque load. FIG. 4D is series of bar graphs showing assessment of plaque count. FIG. 4E is bar graph showing assessment of plaque fold clearance of Aβ CAR-M over control CAR-Ms on APP/PS1 brain slices after co-incubation with no cells, control CAR-M, or Aβ CAR-M with or without GFP-Luc. Data shown as mean±s.e.m from n=5-6 independent experiments with 5-6 technical replicates each. Statistical significance was calculated with one-way ANOVA with Tukey's multiple comparisons test (FIG. 4C (HJ3.4), FIG. 4D) or unpaired t-tests (FIG. 4C (X-34), FIG. 4E). For FIG. 4C-4E, *P<0.05, **P<0.01, ***P<0.001, ****P<0.0001. ns, not significant. Note: 2e, “60-90” only contains 4 datapoints due to a lack of plaques this size in some samples. FIG. 4F is series of representative immunofluorescence microscopy images of individual amyloid-laden control or Aβ CAR-Ms after removal from brain slices from aged APP/PS1 mice for the indicated amount of time and stained with HJ3.4 to visualize intracellular amyloid over time. Scale bars all 2 m. FIG. 4G is a line graph showing quantification of intracellular amyloid in control of Aβ CAR-Ms cultured on brain slices from aged APP/PS1 mice for the indicated amount of time. Data is shown as mean±s.e.m from 18 technical replicates. Each condition was fitted to a one-phase exponential decay function to derive the indicated half-lives.

FIG. 5A-5F show that first generation Aβ CAR-Ms have limited expansion and survival and fail to reduce plaque load in vivo when administered with Busulfan preconditioning. FIG. 5A is a schematic of Busulfan pre-conditioning and intrahippocampal injection of Aβ CAR-Ms. FIG. 5B is a line graph of total flux assessed using non-invasive bioluminescence imaging (BLI) tracking CAR-M persistence after intrahippocampal injection. n=6-14 mice per group. Statistical significance calculated with one-way ANOVA with Tukey's multiple comparisons test. FIG. 5C is series of representative BLI images following Busulfan preconditioning and intrahippocampal injection of Aβ CAR-Ms. Days indicates days post-intrahippocampal injection. FIG. 5D is a representative immunofluorescence microscopy image showing GFP+ CAR-Ms localized to the hippocampus 14 days after intrahippocampal injection. FIG. 5E is representative immunofluorescence microscopy images of Aβ CAR-Ms binding to amyloid plaque in vivo. FIG. 5F is bar graph showing Assessment of plaque load after intrahippocampal injection of PBS or Aβ CAR-Ms in n=6 aged APP/PS1 mice. Mice were sacrificed on day 14 post intrahippocampal injection and brain tissue was sectioned and stained with HJ3.4 to assess plaque load. Data shown as mean±s.e.m. Statistical significance was calculated with unpaired t-tests.

FIG. 6A-6G show that M-CSF reinforced Aβ CAR-Ms have improved survival and reduce plaque load in vivo. FIG. 6A is a schematic of M-CSF expressing construct retrovirally introduced into control and Aβ CAR-Ms, which contains the M-CSF gene followed by a P2A cleavage sequence and Thy1.1. FIG. 6B is a line graph showing % change in live cell count of first-generation Aβ CAR-Ms and M-CSF reinforced Aβ CAR-Ms upon removal of M-CSF from the culture medium in vitro, determined by flow cytometry staining with ZombieNIR live/dead staining. Cells were differentiated for 6 days in M-CSF to become mature macrophages, prior to M-CSF removal. Statistical significance was calculated with an unpaired t-test. FIG. 6C is a schematic of PLX5622 pre-conditioning and intrahippocampal injection of M-CSF reinforced Aβ CAR-Ms. FIG. 6D is a line graph of Total flux determined by non-invasive bioluminescence imaging (BLI) tracking first-generation Aβ CAR-M kinetics in vivo compared to M-CSF reinforced Aβ CAR-M kinetics after intrahippocampal injection. “Days” indicates days post-intrahippocampal injection. n=10-18 mice per group. Statistical significance was calculated with unpaired t-tests. FIG. 6E is a bar graph of fold-expansion of CAR-Ms from the first day of BLI after intrahippocampal injection of cells to the day of maximum total flux measured by BLI. n=6-18 mice per group. Statistical significance was calculated with one-way ANOVA with Tukey's multiple comparisons test. FIG. 6F is a representative immunofluorescence microscopy image of M-CSF reinforced Aβ CAR-Ms binding to amyloid plaque in vivo. FIG. 6G is series of bar graphs of assessment of plaque load after intrahippocampal injection of M-CSF reinforced control CAR-M or M-CSF reinforced Aβ CAR-M in n=12 14-month-old female APP/PS1 mice. Mice were sacrificed on day 12 or 13 post intrahippocampal injection and brain tissue was sectioned and stained with HJ3.4 and X-34 to assess plaque load. Data shown as mean±s.e.m. Statistical significance was calculated with paired t-tests. For FIG. 6B-6D, *P<0.05, **P<0.01, ***P<0.001, ****P<0.0001, ns, not significant.

FIG. 7A-7D show generation and characterization of M-CSF reinforced Aβ CAR-Ms. FIG. 7A is series of representative FACS plots showing surface expression of Thy1.1 on control and Aβ CAR-Ms before and after retroviral transduction of HoxB8 cells with the M-CSF construct and sorting for Thy1.1+ cells. Gated on single, live, CAR+ cells. MFI, mean fluorescence intensity. FIG. 7B is line graph showing % change in live cell count of first-generation Aβ CAR-Ms and M-CSF reinforced Aβ CAR-Ms upon removal of M-CSF from the culture medium in vitro, determined by flow cytometry staining with ZombieNIR live/dead staining. Cells were differentiated for 6 days in M-CSF to become mature macrophages, prior to M-CSF removal. Data is representative of 2 independent experiments with 3 technical replicates per condition per experiment. Statistical significance was calculated with unpaired t-tests. FIG. 7C is series of representative flow cytometry phenotyping of M-CSF reinforced Aβ CAR-Ms after differentiating with M-CSF for 6 days in vitro. Data are representative of >3 independent experiments. FIG. 7D is a bar graph showing quantification of Cell Trace Violet (CTV) dilution in the stated conditions as the % of macrophages that diluted CTV 72 hrs after staining. Statistical significance was calculated with a one-way ANOVA with Tukey's multiple comparisons test, and data is representative of two independent experiments with three technical replicates per condition per experiment. All data shown as mean±s.e.m. *P<0.05, **P<0.01, ***P<0.001, ****P<0.0001, ns not significant.

FIG. 8A-8B show M-CSF reinforced Aβ CAR-Ms expand in vivo when administered with PLX5622 preconditioning. FIG. 8A is series of representative bioluminescence images following PLX5622 preconditioning and intrahippocampal injection of M-CSF reinforced Aβ CAR-Ms. Days indicates days post-intrahippocampal injection. FIG. 8B is representative image showing the six regions of interest (ROI) in which Iba-1 positive cells were quantified in each hippocampus and a higher power view of one ROI showing the cell masking used for quantification (left upper panel), representative images showing one ROI from mice treated with PBS for 2 days, 20 mg/kg Busulfan for 2 days, or 50 mg/kg PLX5622 for 4 days, twice a day (left lower panel), and quantification of Iba-1+ cells where each dot represents the average Iba-1+ cell count in all ROIs per mouse (right panel). Statistical significance was calculated with a one-way ANOVA with Tukey's multiple comparisons test.

FIG. 9A-9D show that M-CSF reinforced Aβ CAR-Ms reduce plaque load in the locally in the hippocampus in vivo. FIG. 9A is series of representative images of brain sections from reinforced CAR-M treated aged APP/PS1 mice stained with HJ3.4. Images indicate circular regions of interest centered around GFP signal in the hippocampus representing the cell injection area in which plaque was quantified, highlighted in higher magnification on the right. Control regions of interest were quantified in the cortex to ensure uniform plaque load between mice. “C”=reinforced control CAR-M treated side, “E”=reinforced Aβ CAR-M treated side. FIG. 9B is series of bar graphs showing assessment of plaque load in the cortex and the hippocampus (HPC) after intrahippocampal injection of M-CSF reinforced control or Aβ CAR-Ms in n=12, 14-month-old female APP/PS1 mice. Mice were sacrificed on day 12 or 13 post intrahippocampal injection and brain tissue was sectioned and stained with HJ3.4 or X-34 to assess plaque load in the regions of interest shown in FIG. 9A. FIG. 9C-9D are series of plaque load data from control or Aβ CAR-M treated mice in FIG. 6G here stratified by sex, shown for HJ3.4 in FIG. 9C and X-34 in FIG. 9D per brain slice, per mouse, and comparing the cortex to the hippocampus (HPC). Data shown as mean±s.e.m. Statistical significance was calculated with unpaired t-tests for brain slice data and paired t-tests for per mouse data. *P<0.05, **P<0.01, ***P<0.001, ****P<0.0001, ns, not significant.

### DETAILED DESCRIPTION

The current disclosure is based partly on the unexpected finding that introducing a CAR into macrophages that targets Aβ and comprises the phagocytic common gamma chain of the Fc receptor (FcRγ) as an intracellular signaling domain effectively induces Aβ endocytosis in vitro and in vivo. The inventors engineered a first-generation CAR-M that resorbs soluble Aβ and amyloid plaques in vitro and resorbs and degrades Aβ plaques from APP/PS1 brain slices ex vivo. The current disclosure further provides a next-generation reinforced CAR-M that secretes a cytokine, for example macrophage colony-stimulating factor (M-CSF), to facilitate its own survival. The reinforced CAR-M significantly expands in the brain microenvironment after microglia depletion with the CSF-1 inhibitor PLX5622. Furthermore, the cytokine (for example, M-CSF) reinforced CAR-Ms significantly resorbed plaques locally in the hippocampi of aged APP/PS1 mice compared to control M-CSF secreting CAR-Ms targeting an irrelevant antigen. The CAR-M disclosed herein may be used for reducing amyloid plaque load and treating AD.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6, 9, and 7.0 are explicitly contemplated.

The use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

The terms “nucleic acid,” “nucleic acid molecule,” and “polynucleotide” are used interchangeably herein. The terms “nucleic acid encoding . . . ” or “nucleic acid molecule encoding . . . ” should be understood as referring to the sequence of nucleotides which encodes a polypeptide. Deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Within the context of the application a protein is represented by an amino acid sequence and correspondingly a nucleic acid molecule or a polynucleotide represented by a nucleic acid sequence. Identity and similarity between sequences: throughout this application, each time one refers to a specific nucleic acid sequence SEQ ID NO (take SEQ ID NO: Y as example), one may replace it by a polynucleotide represented by a nucleic acid sequence comprising a sequence that has at least 60%, 70%, 75%, 80%, 85%, 90%, 95, or 99% sequence identity or similarity with nucleic acid SEQ ID NO: Y.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of or reducing one or more symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system and “exogenous” refers to any material introduced from or produced outside an organism, cell, tissue or system.

As used herein, the term “antibody” is intended to denote an immunoglobulin molecule that possesses a “variable region” antigen recognition site. The term “variable region” is intended to distinguish such domain of the immunoglobulin from domains that are broadly shared by antibodies (such as an antibody Fc domain). The variable region comprises a “hypervariable region” whose residues are responsible for antigen binding. The hypervariable region comprises amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelid antibodies, single chain antibodies, disulfide-linked Fvs (sdFv), intrabodies, and anti-idiotypic (anti-Id) antibodies (including, e.g., anti-Id and anti-anti-Id antibodies to antibodies of the invention). In particular, such antibodies include immunoglobulin molecules of any type (e.g., IgG, IgE, IgM, IgD, IgA and IgY), class (e.g., IgG1, IgG2, IgG3, IgG4, IgA1 and IgA2) or subclass.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments and human and humanized versions thereof.

As used herein, the term “antigen binding fragment” of an antibody refers to one or more portions of an antibody that contain the antibody's CDR and optionally the framework residues that comprise the antibody's “variable region” antigen recognition site, and exhibit an ability to immunospecifically bind antigen. Such fragments include Fab′, F(ab′)₂, Fv, single chain (ScFv), and mutants thereof, naturally occurring variants, and fusion proteins comprising the antibody's “variable region” antigen recognition site and a heterologous protein (e.g., a toxin, an antigen recognition site for a different antigen, an enzyme, a receptor or receptor ligand, etc.). As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues.

Single-chain forms of antibodies, and their higher order forms, may include, but are not limited to, single-domain antibodies, single chain variant fragments (scFvs), divalent scFvs (di-scFvs), trivalent scFvs (tri-scFvs), tetravalent scFvs (tetra-scFvs), diabodies, and triabodies and tetrabodies. ScFv's are comprised of heavy and light chain variable regions connected by a linker. In most instances, but not all, the linker may be a peptide. A linker peptide is preferably from about 5 to 30 amino acids in length, or from about 10 to 25 amino acids in length. Typically, the linker allows for stabilization of the variable domains without interfering with the proper folding and creation of an active binding site. In preferred embodiments, a linker peptide is rich in glycine, as well as serine or threonine. ScFvs can be used to facilitate phage display or can be used for flow cytometry, immunohistochemistry, or as targeting domains. Methods of making and using scFvs are known in the art. ScFvs may also be conjugated to a human constant domain (e.g. a heavy constant domain is derived from an IgG domain, such as IgG1, IgG2, IgG3, or IgG4, or a heavy chain constant domain derived from IgA, IgM, or IgE). Diabodies, triabodies, and tetrabodies and higher order variants are typically created by varying the length of the linker peptide from zero to several amino acids. Alternatively, it is also well known in the art that multivalent binding antibody variants can be generated using self-assembling units linked to the variable domain.

As used herein “humanized antibody” refers to a non-human antibody that has been modified to reduce the risk of the non-human antibody eliciting an immune response in humans following administration but retains similar binding specificity and affinity as the starting non-human antibody. A humanized antibody binds to the same or similar epitope as the non-human antibody. The term “humanized antibody” includes an antibody that is composed partially or fully of amino acid sequences derived from a human antibody germline by altering the sequence of an antibody having non-human hypervariable regions (“HVR”). The simplest such alteration may consist simply of substituting the constant region of a human antibody for the murine constant region, thus resulting in a human/murine chimera which may have sufficiently low immunogenicity to be acceptable for pharmaceutical use. Preferably, the variable region of the antibody is also humanized by techniques that are by now well known in the art. For example, the framework regions of a variable region can be substituted by the corresponding human framework regions, while retaining one, several, or all six non-human HVRs. Some framework residues can be substituted with corresponding residues from a non-human VL domain or VH domain (e.g., the non-human antibody from which the HVR residues are derived), e.g., to restore or improve specificity or affinity of the humanized antibody. Substantially human framework regions have at least about 75% homology with a known human framework sequence (i.e. at least about 75%, at least about 80%, at least about 85%, at least about 90%, at least about 95%, or at least about 99% sequence identity). HVRs may also be randomly mutated such that binding activity and affinity for the antigen is maintained or enhanced in the context of fully human germline framework regions or framework regions that are substantially human. As mentioned above, it is sufficient for use in the methods of this discovery to employ an antibody fragment. Further, as used herein, the term “humanized antibody” refers to an antibody comprising a substantially human framework region, at least one HVR from a nonhuman antibody, and in which any constant region present is substantially human. Substantially human constant regions have at least about 90% with a known human constant sequence (i.e. about 90%, about 95%, or about 99% sequence identity). Hence, all parts of a humanized antibody, except possibly the HVRs, are substantially identical to corresponding pairs of one or more germline human immunoglobulin sequences.

As used herein, “expression” refers to the transcription and/or translation of a particular nucleotide sequence driven by its promoter.

A “vector” is a nucleic acid sequence designed to be propagated and or transcribed upon exposure to a cellular environment, such as a cell lysate or a whole cell. Vector: A vector may comprise a polynucleotide cassette as defined herein. A vector as described herein may be selected from any genetic element known in the art which can facilitate transfer of nucleic acids between cells, such as, but not limited to, plasmids, transposons, cosmids, chromosomes, artificial chromosomes, viruses, virions, and the like. A vector may also be a chemical vector, such as a lipid complex or naked DNA. “Naked DNA” or “naked nucleic acid” refers to a nucleic acid molecule that is not contained in encapsulating means that facilitates delivery of a nucleic acid into the cytoplasm of a target host cell. Naked DNA may be circular or linear (linearized DNA sequence). Optionally, a naked nucleic acid can be associated with standard means used in the art for facilitating its delivery of the nucleic acid to the target host cell, for example to facilitate the transport of the nucleic acid through the cell membrane. A vector may be a viral vector.

As used herein, the term “viral vector” can refer to a nucleic acid vector construct that includes at least one element of viral origin and has the capacity to be packaged into a viral vector particle and encodes at least an exogenous polynucleotide. A viral vector is a modified virus that serves as a delivery vehicle for the transfer of genetic material into a host cell. The viral vector is engineered to carry and express a specific gene or genes of interest in the target cells. The viral vector retains the ability to infect cells but is modified to be replication-deficient or replication-competent with controlled replication, ensuring safety and controllability. In certain aspects, the vector and/or particle can be utilized for the purpose of transferring any nucleic acids into cells either in vitro or in vivo. Numerous viral vectors are known in the art. The term virion can refer to a single infective viral particle. “Viral vector,” “viral vector particle,” and “viral particle” also refer to a complete virus particle with its DNA or RNA core and protein coat as it exists outside the cell. Non-limiting examples of viral vectors for use herein can include adenoviruses, adeno-associated viruses (AAV), herpesviruses, retroviruses, lentiviruses, integrase defective lentiviruses (IDLV), and the like.

As used herein, “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g, naked or contained in liposomes) and viruses (e.g., lentiviruses, retroviruses, adenoviruses (e.g., Ad5F35), and adeno-associated viruses) that incorporate the recombinant polynucleotide.

As used herein, “chimeric antigen receptor” or “CAR,” refers to an artificial T cell surface receptor that is engineered to be expressed on an immune effector cell and specifically targets a cell and/or binds an antigen. CARs may be used as a therapy with adoptive cell transfer. Monocytes, macrophages and/or dendritic cells are removed from a patient (e.g., via blood or ascites fluid) and modified so that they express the receptors specific to a particular form of antigen. The CARs may have been expressed with specificity for amyloid protein antigens, for example. The CARs may also comprise an intracellular activation domain, a transmembrane domain and an extracellular domain comprising, for example, an amyloid protein antigen binding region. In one example, CARs may comprise fusions of single-chain variable fragments (scFv) derived monoclonal antibodies, FcRγ transmembrane domains and intracellular domains. The specificity of CAR designs may be derived from ligands of receptors (e.g., peptides). For example, a CAR can target a neurodegenerative disease/disorder by redirecting an immune cell, for example a macrophage, expressing the CAR specific for protein aggregates, associated with the disease/disorder.

The term “neurodegenerative disease” as used herein, refers to a neurological disease characterized by loss or degeneration of neurons and/or by the presence of misfolded protein aggregates in the cytoplasm and/or nucleus of nerve cells or in the extracellular space. Neurodegenerative diseases include neurodegenerative movement disorders and neurodegenerative conditions relating to memory loss and/or dementia. Neurodegenerative diseases include tauopathies and a-synucleopathies. Examples of neurodegenerative diseases include, but are not limited to, presenile dementia, senile dementia, Alzheimer's disease, progressive supranuclear palsy (PSP), Pick's disease, primary progressive aphasia, frontotemporal dementia, corticobasal dementia, Parkinson's disease, Parkinson's disease with dementia, dementia with Lewy bodies, Down's syndrome, multiple system atrophy, amyotrophic lateral sclerosis (ALS) and Hallervorden-Spatz syndrome.

As used herein “binds,” refers to a polypeptide (including antibodies) or receptor, binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an antigen) when it does not bind in a significant amount to other proteins present in the sample or to other proteins to which the ligand or antibody may come in contact in an organism.

As used herein, “amyloid protein” is intended to denote a protein which is involved in the formation of fibrils, plaques and/or amyloid deposits, either by being part of the fibrils, plaques and/or deposits as such or by being part of the biosynthetic pathway leading to the formation of the fibrils, plaques and/or amyloid deposits. In the present context the term “protein” or is intended to mean both short peptides of from 2 to 10 amino acid residues, oligopeptides of from 11 to 100 amino acid residues, polypeptides of more than 100 amino acid residues, and full length proteins. The terms also encompass peptides having substantial similarity to amyloid proteins, such as, e.g., structural variants. In some cases, the proteins occur naturally or be synthetically constructed. The term amyloid protein or amyloid like protein also includes amyloidgenic proteins and proteins that produce amyloid like morphology.

As used herein, “Amyloid beta,” “Aβ,” “Abeta,” or “beta-amyloid” can be used interchangeably and may refer to peptides with 36-43 amino acids that are the main component of the amyloid plaques in subjects with neurodegenerative disease such as Alzheimer's disease. The peptides derive from the amyloid-beta precursor protein (APP), which is cleaved by beta secretase and gamma secretase to yield Aβ in a cholesterol-dependent process and substrate presentation. Both neurons and oligodendrocytes produce and release Aβ in the brain, contributing to formation of amyloid plaques. As molecules can aggregate to form flexible soluble oligomers which may exist in several forms. It is now believed that certain misfolded oligomers (known as “seeds”) can induce other Aβ molecules to also take the misfolded oligomeric form, leading to a chain reaction akin to a prion infection. The oligomers are toxic to nerve cells. The other protein implicated in neurodegenerative disease such as Alzheimer's disease, tau protein, may also form such prion-like misfolded oligomers, and there is some evidence that misfolded Aβ may induce tau to misfold.

### 2. Chimeric Antigen Receptor (CAR)

Provided herein is a chimeric antigen receptor (CAR) comprising an antigen binding domain that specifically binds to beta amyloid (Aβ). The CAR may comprise a signal peptide, the antigen binding domain, a hinge region, a transmembrane domain, at least one co-stimulatory domain, and a signaling domain. The hinge region and the antigen binding domain may be collectively referred to as the extracellular domain. The at least one co-stimulatory domain and signaling domain may be collectively referred to as the intracellular domain. In one example, the CAR may comprise the antigen binding domain, a linker, a transmembrane domain, and an intracellular domain.

The signal peptide directs the transport of any secreted or transmembrane protein to the cell membrane and cell surface allowing correct localization of the polypeptide. In particular, the signal peptide of the present disclosure may direct the polypeptide to the cellular membrane, wherein the extracellular portion, i.e. antigen recognition domain or target element of the polypeptide is displayed on the cell surface, the transmembrane portion spans the plasma membrane, and the signaling domain is in the cytoplasmic portion, or interior of the cell. The signal peptide may include an antibody light chain signal sequence, an antibody heavy chain signal sequence, and other signal sequences know in the art (e.g., see Haryadi et al. PLoS One. 2015 Feb. 23; 10(2):e0116878). The signal peptides may have 16 to 30 amino acids and may comprise of three regions of the basic N-terminal region, a central hydrophobic region, and a more polar C-terminal region. In one instance, the signal peptide may comprise an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 20.

The antigen binding domain or target element of the CAR includes a polypeptide that is selective for or targets an antigen, receptor, peptide ligand, protein ligand of the target, or a polypeptide of the target.

The antigen binding domain of the CAR recognizes and specifically binds an antigen. The antigen binding domain may specifically bind an antigen when, for example, it binds the antigen with an affinity constant or affinity of interaction (KD) between about 0.1 μM to about 10 μM, preferably about 0.1 μM to about 1 μM, more preferably about 0.1 μM to about 100 nM. Methods for determining the affinity of interaction are well-known in the art. The antigen binding domain may be any antigen-binding polypeptide, a wide variety of which are well-known in the art. Non-limiting examples include a full-length antibody, an antigen-binding fragment, a Fab, a single-chain variable fragment (scFv), or a single-domain antibody. The antibody may be a monoclonal antibody, a polyclonal antibody, a synthetic antibody, a human antibody, a humanized antibody, a non-human antibody, and any fragment thereof. In some instances, the antigen-binding domain is a single chain Fv (scFv). The scFv may be expressed on the surface of a CAR expressing cell and confers antigen specificity. The scFv is derived from the portion of an antibody that specifically recognizes a target protein. Other antibody-based recognition domains (cAb VHH (camelid antibody variable domains) and humanized versions thereof, lgNAR VH (shark antibody variable domains) and humanized versions thereof, sdAb VH (single domain antibody variable domains) and “camelized” antibody variable domains are suitable for use. In some instances, T-cell receptor (TCR) based recognition domains such as single chain TCR (scTv, single chain two-domain TCR containing VaVP) are also suitable for use. In one example, the CAR may comprise antigen binding domain comprising a scFv.

The antigen binding domain of the CAR may bind for example, a protein aggregate. The protein aggregate may comprise two or more proteins (e.g., two or more of the same protein, two or more different proteins, etc.) that have aggregated together in a tissue in a subject to give rise to a pathological condition, or which places the subject at risk for a pathological condition. The protein aggregate may be or comprise one or more of misfolded protein(s), otherwise improperly formed/malformed protein(s), for e.g., as a result of a mutation which may not affect folding but does affect function, and/or an aggregation of protein and non-protein components, for e.g., nucleic acids, small molecules, etc. Non-limiting examples of such protein aggregates include aggregates of amyloid protein, aggregates of tau protein, aggregates of TDP-43 protein, aggregates of immunoglobulin light chains or transthyretin protein, aggregates of prion protein and the like.

For example, the antigen binding domain of the CAR may specifically bind to beta amyloid (Aβ). The Aβ may be an aggregated form of Aβ. The antigen binding domain of the CAR may bind to oligomers and fibrils of Aβ. In one instance, the antigen binding domain may comprise an anti-Aβ antibody or an antigen binding fragment thereof. Non-limiting examples of the anti-Aβ antibody include Bapineuzumab, Solanezumab, Gantenerumab, Crenezumab, Ponezumab, BAN2401, Aducanumab, and Donenemab.

The CAR may comprise an antigen binding domain comprising a scFv of Aducanumab. In such instances, the antigen binding domain may comprise a heavy chain variable region (VH) comprising an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 1. The antigen binding domain may further comprise a light chain variable region (VL) comprising an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 2.

In yet another example, the CAR may comprise an antigen binding domain comprising a scFv of Donenemab. In such instances, the antigen binding domain may comprise a VH comprising an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 15. The antigen binding domain may further comprise a VL comprising an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 16.

The VH and VL of the scFv may be directly joined or joined by a linker. The linker may link VH with the VL of the antigen binding domain. The linker may be a polypeptide between 2 and 50 amino acids in length. Various linker sequences are known in the art, including, without limitation, glycine serine linkers. In one instance the linker may comprise the amino acid sequence set forth in SEQ ID NO: 9 or an amino acid sequence having at least about 85%, 90%, 95%, or 99% sequence identity thereto.

In one example, the scFv with VH and VL joined by linker may comprise the an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in any one of SEQ ID NO: 18 or SEQ ID NO: 19.

The antibody may further be a bispecific antibody, a multi-specific antibody, or a hybrid antibody having at least two distinct antigen binding domains with different specificities, with at least one antigen binding domain comprising an antibody that specifically binds to Aβ or a fragment thereof, disclosed herein. Such antibody constructs may be recombinant protein constructs made from two flexibly linked antibody derived binding domains, with a first antigen binding domain specific for Aβ and at least a second antigen binding domain that binds to engages, and/or activates immune cells, for example macrophage cells. The second antigen binding domain may comprise an antibody or fragments thereof that binds to OX40, CD40, CD70, TLR4, TLR9, CD47 or a STING agonist. In another example, the bispecific antibody, a multi-specific antibody, or a hybrid antibody may comprise a first antigen binding domain specific for Aβ as disclosed herein and at least a second antigen binding domain that binds to tau. Non-limiting examples of anti-tau antibodies include Gosuranemab, Tilavonemab, Bepranemab, Zagotenemab, Semorinemab, RG7345, JNJ-63733657, BIIB076, E2814, Lu AF87908, and PNT001

The CAR may further comprise a linker that links the transmembrane and intracellular domains to the antigen binding domain. The linker may be a polypeptide between 2 and 50 amino acids in length. Suitable linkers are known in the art and any linker to achieve a desired flexibility, spatial organization, and proximity may be used. In one instance, the linker may comprise the amino acid sequence set forth in SEQ ID NO: 4 or an amino acid sequence having at least about 85%, 90%, 95%, or 99% sequence identity thereto.

The transmembrane domain of the CAR may connect the antigen binding domain to the intracellular domain. A transmembrane domain may be derived either from a natural or from a synthetic source. The transmembrane domain may be derived from any membrane-bound or transmembrane protein. For example, transmembrane domain may be derived from the alpha, beta or zeta chain of the T-cell receptor, CD28, FcRγ, FcR beta, CD3 gamma, CD3 delta, CD3 epsilon, CD45, CD4, CD5, CD8, CD9, CD16, CD22, CD33, CD37, CD64, CD80, CD86, CD134, CD137, CD154, Toll-like receptor 1 (TLR1), TLR2, TLR3, TLR4, TLR5, TLR6, TLR7, TLR8, and TLR9. The transmembrane domain may further comprise one or more hinge regions. In some instances, any of a variety of human hinge regions can be employed, for e.g., a CD28 or CD8 hinge region, including the human Ig (immunoglobulin) hinge region.

In one example, the transmembrane domain of the CAR may comprise one or more regions derived from FcRγ. The FcRγ may be a murine, a humanized, or a human FcRγ. In one instance, the CAR may comprise a transmembrane derived from murine FcRγ comprising an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 5. Alternatively, the CAR may comprise a transmembrane derived from human FcRγ comprising an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 7.

The CAR of the present disclosure may also comprise an intracellular domain. Examples of an intracellular domain for use include, but are not limited to, the cytoplasmic portion of a surface receptor, co-stimulatory molecule, and any molecule that acts in concert to initiate signal transduction in a monocyte, macrophage or dendritic cell, as well as any derivative or variant of these elements and any synthetic sequence that has the same functional capability. The intracellular domain may comprise a fragment or domain from one or more molecules or receptors including, but are not limited to, FcRγ, TCR, CD3 zeta, CD3 gamma, CD3 delta, CD3 epsilon, CD86, FcR beta (Fc Epsilon Rib), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CDIIc, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combinations thereof.

In one instance, the intracellular domain may comprise one or more regions derived from FcRγ. The FcRγ may be a murine, a humanized, or a human FcRγ. In one instance, the CAR may comprise a intracellular domain derived from murine FcRγ comprising an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 6. Alternatively, the CAR may comprise a intracellular domain derived from human FcRγ comprising an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 8. In one example, both the transmembrane domain and the intracellular domain are murine or human.

The CAR may comprise an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in any one of SEQ ID NO: 11 or SEQ ID NO: 21. In an alternative instance, the CAR may comprise an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in any one of SEQ ID NO: 12 OR SEQ ID NO: 22.

### 3. Vector

Further provided herein are a nucleic acid encoding the CAR and a vector comprising the nucleic acid. The vector may be a plasmid vector, viral vector, retrotransposon, a phagemid, a phage derivative, an animal virus, a cosmid, a site directed insertion vector (e.g. CRISPR, Zn finger nucleases, TALEN), or suicide expression vector. Alternatively, a polynucleotide, for example, an mRNA, encoding the CAR may be introduced into a cell using physical or chemical means. Physical methods for introducing a polynucleotide into a host cell include calcium phosphate precipitation, lipofection, particle bombardment, microinjection, electroporation, and the like. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo may be a liposome (e.g., an artificial membrane vesicle).

In one example, the vector may be a viral vector. Viral vector for use herein may include Moloney Murine Leukemia virus (MuLV), other retroviruses, lentiviruses, adenoviruses, adeno-associated viruses, or herpes viruses. The vector, in one instance, may be MuLV. The vector may be used to introduce the CAR into an immune cell. The vector may further comprise other regulatory elements. The vector may contain transcription and translation terminators, initiation sequences, and promoters useful for regulation of the expression of the CAR nucleic acid sequence. The vector may further comprise an origin of replication functional in at least one organism, a promoter sequence, convenient restriction endonuclease sites, and one or more selectable markers.

The vector may further comprise a reporter gene. The reporter gene may facilitate identification and selection of expressing cells from the population of cells sought to be transfected or infected. In another instance the reporter, may facilitate in vivo or in vitro imaging for assessing the progress of treatment with the CAR described herein. The reporter gene may encode a fluorescent protein. The fluorescent protein may be any one of tdTomato, GFP (green fluorescent protein), eGFP, BFP (blue fluorescent protein), YFP (yellow fluorescent protein), RFP (red fluorescent protein), mCherry, luciferase, or any derivative thereof.

### 4. CAR Macrophage (CAR-M)

Described herein is a cell expressing the CAR. Methods of delivering a vector encoding the CAR and obtaining a population of cells expressing the CAR are known in the art. An exemplary method of generating cells comprising the CAR is provided herein below. The cell may be an immune cell, for example a macrophage. In such instances, macrophages may be modified to express the CAR (CAR-M). Macrophages may be obtained naturally or commercially. Macrophages may be obtained from an animal or a human. Macrophages may be isolated from tissues, non-limiting examples of which include blood, umbilical cord, bone marrow, lungs, and peritoneum. Any known method of isolation such as magnetic bead-conjugated antibody cell isolation, density gradient separation, fluorescence-activated cell sorting (FACS), or adhesion-mediated purification may be used. Alternatively, precursor cells such as myeloid precursors or hematopoietic stem cells may be in vitro or ex vivo differentiated into macrophages. Macrophages or precursor cells may be obtained autologously or sourced from allogeneic or universal donors. In one example, precursor cells such as myeloid precursors or hematopoietic stem cells may first modified to express the CAR described herein, and then differentiated into macrophages in vitro or ex vivo.

The cell expressing the CAR may further be modified to express a cytokine. The cytokine may be introduced into the cell using a vector. The vector may be a plasmid vector, viral vector, retrotransposon, a phagemid, a phage derivative, an animal virus, a cosmid, a site directed insertion vector (e.g. CRISPR, Zn finger nucleases, TALEN), or suicide expression vector. Alternatively, a polynucleotide, for example, an mRNA, encoding the cytokine may be introduced into a cell using physical or chemical means. Physical methods for introducing a polynucleotide into a host cell include calcium phosphate precipitation, lipofection, particle bombardment, microinjection, electroporation, and the like. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo may be a liposome (e.g., an artificial membrane vesicle). In one instance, the cell expressing CAR may be modified to express a cytokine using a vector. The vector may be the same vector comprising a nucleic acid sequence encoding the CAR. In such instances, the polynucleotide encoding the CAR may be separated using a 2A site or an internal ribosome entry site (IRES) from the polynucleotide encoding the cytokine. Alternatively, the cytokine may be expressed by delivering a second vector expressing a polynucleotide encoding the cytokine. In such instances, the cell comprises a first vector encoding the CAR and a second vector encoding the cytokine. The second vector may be the same or different type of vector as the first vector.

The cell expressing CAR further modified to express the cytokine, for example a CAR-M, may exhibit enhanced persistence or survival, when administered in a subject, compared to a cell expressing the CAR but not the cytokine. For example, the cell expressing CAR further modified to express the cytokine may persist or survive at least 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, or more compared to a cell expressing only the CAR.

The cytokine may be a secreted cytokine and may be a murine, an animal, a humanized, or a human cytokine. In one instance, the cytokine may be macrophage colony-stimulating factor (M-CSF) or Granulocyte-macrophage colony-stimulating factor (GM-CSF). For example, the cytokine may be murine M-CSF and may comprise an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 13. In another instance, the cytokine may be human M-CSF and may comprise an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 14. In yet another instance, the cytokine may be human GM-CSF and may comprise an amino acid sequence at least about 80% or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to the amino acid sequence set forth in SEQ ID NO: 17.

### 5. Composition

Further provided herein is a composition comprising the cell expressing the CAR, optionally further modified to express a cytokine. The composition may be formulated as a pharmaceutical composition. The pharmaceutical composition may comprise a vector, a cell, or a population of cells as defined earlier herein, preferably for use as a medicament. For example, the pharmaceutical composition may comprise the CAR-M cells disclosed herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. The compositions may further include other components such as IL-2, IL-15, other cytokines or cell populations, or components that sustain the viability and/or activity of the CAR expressing cells. The CAR expressing cells may be formulated as a single dosage unit or as multiple dosage units.

Pharmaceutically acceptable diluents, carriers, and excipients may include, but are not limited to, physiological saline, Ringer's solution, phosphate solution or buffer, buffered saline, and other carriers known in the art. The term “carrier” refers to a diluent, adjuvant (e.g., Freund's adjuvant (complete and incomplete), excipient, or vehicle with which the therapeutic is administered. Such pharmaceutical carriers may be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Other pharmaceutically acceptable carriers include any and all solvents, adjuvants, dispersion media, coatings, surfactants, antioxidants, preservatives (e.g., antibacterial agents, antifungal agents), isotonic agents, absorption delaying agents, salts, drugs, drug stabilizers, gels, binders, excipients, disintegration agents, lubricants, sweetening agents, flavoring agents, dyes, colorants, other medicinal or pharmaceutical agents, wetting agents, emulsifying agents, solution promoters, solubilizers, antifoaming agents, and such like materials and any combinations thereof, as would be known to one of ordinary skill in the art. Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may also be found for example in Remington's Pharma. Sci. 18th ed. 1990. Except insofar as any conventional carrier is incompatible with the active ingredient, its use in the therapeutic or pharmaceutical compositions is contemplated.

The compositions may take the form of solutions, suspensions, emulsion, tablets, pills, capsules, powders, sustained-release formulations and the like. Generally, the ingredients of the pharmaceutical composition may be supplied either separately or mixed together in unit dosage form, for example, as a dry lyophilized powder or water free concentrate in a hermetically sealed container such as an ampoule or sachette indicating the quantity of active agent. Where the pharmaceutical composition is to be administered by infusion, it can be dispensed with an infusion bottle containing sterile pharmaceutical grade water or saline. Where the pharmaceutical composition is administered by injection, an ampoule of sterile water for injection or saline may be provided so that the ingredients may be mixed prior to administration. The pharmaceutical composition may be formulated as neutral or salt forms. Pharmaceutically acceptable salts include, but are not limited to, those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The composition comprising the CAR expressing cells disclosed herein, may be formulated for administration, in any convenient manner, including for injection, transfusion, or implantation. The compositions may be formulated for subcutaneous, intradermal, intracranial, intranodal, intramedullar, intramuscular, intravenous (i.v.), or intraperitoneal administration. For example, the compositions may be formulated for intracranial or intraperitoneal administration. Administration may be systemic or local.

The composition may comprise from about 1×10²/μl to 5×10¹/μl of the CAR expressing cells. For example, the composition may comprise 1×10²/μl, 1×10³/μl, 1×10⁴/μl, 1×10⁵/μl, 1×10⁶/μl, 1×10⁷/μl, 1×10⁸/μl, 1×10⁹/μl, 1×10¹⁰/μl, 1.5×10²/μl, 1.5×10³/μl, 1.5×10⁴/μl, 1.5×10⁵/μl, 1.5×10⁶/μl, 1.5×10⁷/μl, 1.5×10⁸/μl, 1.5×10⁹/μl, 1.5×10¹⁰/μl, 2×10²/μl, 2×10³/μl, 2×10⁴/μl, 2×10⁵/μl, 2×10⁶/μl, 2×10⁷/μl, 2×10⁸/μl, 2×10⁹/μl, 2×10¹⁰/μl, 2.5×10²/μl, 2.5×10³/μl, 2.5×10⁴/μl, 2.5×10⁵/μl, 2.5×10⁶/μl, 2.5×10⁷/μl, 2.5×10⁸/μl, 2.5×10⁹/μl, 2.5×10¹⁰/μl, 3×10²/μl, 3×10³/μl, 3×10⁴/μl, 3×10⁵/μl, 3×10⁶/μl, 3×10⁷/μl, 3×10⁸/μl, 3×10⁹/μl, 3×10¹⁰/μl, 3.5×10²/μl, 3.5×10³/μl, 3.5×10⁴/μl, 3.5×10⁵/μl, 3.5×10⁶/μl, 3.5×10⁷/μl, 3.5×10⁸/μl, 3.5×10⁹/μl, 3.5×10¹⁰/μl, 4×10²/μl, 4×10³/μl, 4×10⁴/μl, 4×10⁵/μl, 4×10⁶/μl, 4×10⁷/μl, 4×10⁸/μl, 4×10⁹/μl, 4×10¹⁰/μl, 4.5×10²/μl, 4.5×10³/μl, 4.5×10⁴/μl, 4.5×10⁵/μl, 4.5×10⁶/μl, 4.5×10⁷/μl, 4.5×10⁸/μl, 4.5×10⁹/μl, 4.5×10¹/μl, 5×10²/μl, 5×10²/μl, 5×10⁴/μl, 5×10⁵/μl, 5×10⁶/μl, 5×10⁷/μl, 5×10⁸/μl, 5×10⁹/μl, or 5×10¹⁰/μl of the cells.

The composition may further comprise one or more active agents in addition to the CAR, vectors, cells and/or populations of cells provided herein. Non limiting examples of additional active agents include but are not restricted to antibiotics, anti-pyrectics, antimicrobials, antifungals, NSAIDs, and drugs related to treatment of neurodegenerative disorder, such as Alzheimer's disease. For example, the composition may further comprise a cholinesterase inhibitor, an N-methyl D-aspartate (NMDA) antagonist, an antidepressant, a gamma-secretase inhibitor, a beta-secretase inhibitor, an anti-Aβ antibody, an anti-tau antibody, an antagonist of the serotonin receptor 6, a p38alpha MAPK inhibitor, recombinant granulocyte macrophage colony-stimulating factor, a passive immunotherapy, an active vaccine, a tau protein aggregation inhibitor, an anti-inflammatory agent, a phosphodiesterase 9A inhibitor, a sigma-1 receptor agonist, a kinase inhibitor, a phosphatase activator, a phosphatase inhibitor, an angiotensin receptor blocker, a CB1 and/or CB2 endocannabinoid receptor partial agonist, a J-2 adrenergic receptor agonist, a nicotinic acetylcholine receptor agonist, a 5-HT2A inverse agonist, an alpha-2c adrenergic receptor antagonist, a 5-HT 1A and 1D receptor agonist, a glutaminyl-peptide cyclotransferase inhibitor, a selective inhibitor of APP production, a monoamine oxidase B inhibitor, a glutamate receptor antagonist, an AMPA receptor agonist, a nerve growth factor stimulant, a HMG-CoA reductase inhibitor, a neurotrophic agent, a muscarinic M1 receptor agonist, a GABA receptor modulator, a PPAR-gamma agonist, a microtubule protein modulator, a calcium channel blocker, an antihypertensive agent, a statin, or any combinations thereof.

The actual dosage amount of a composition according to the present disclosure, and the selection of any combination treatment to be administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject

### 6. Method of Treatment

The CAR, compositions described herein may be used to treat, prevent, or reduce one or more symptoms associated with Alzheimer's disease. Provided herein is a method of use in a subject in need thereof, which may comprise administering the cells expressing the CAR to the subject, who may be in need thereof. The symptoms may include diffuse or compact Aβ plaques in the subject. The method may reduce the diffuse or compact Aβ plaques by at least 10%, e.g., 20%, 30%, 40%, 50%, 60%, 70%, 80%, 90%, 95% or above, as compared to one or more control subjects not receiving the treatment. Amyloid plaque may be assessed using technique known in the art, such as for example, positron emission tomography (PET) imaging, radiotracers, and magnetic resonance imaging (MRI).

The method may comprise reducing Aβ plaques. In such instances, the method may induce resorption or degradation of Aβ plaques. Alternatively, the method may reduce plaque load. For example, the method may induce resorption or degradation of Aβ plaques, or reduce plaque load by at least 10%, e.g., 20%, 30%, 40%, 50%, 60%, 70%, 80%, 90%, 95% or above, as compared to a subject not receiving the treatment. Amyloid plaques may be assessed using technique known in the art, such as for example, positron emission tomography (PET) imaging, radiotracers, or magnetic resonance imaging (MRI).

The administration of a cell expressing the CAR may be carried out systemically or locally, through injection, transfusion, or implantation. Methods of administration for use include subcutaneous, intradermal, intracranial, intranodal, intramedullar, intramuscular, intravenous (i.v.), or intraperitoneal administration. In one instance, the methods may comprise intracranial or intraperitoneal administration.

The CAR compositions disclosed herein may be used in combination with one or more other therapies including but not limited to antibiotics, anti-pyrectics, antimicrobials, antifungals, NSAIDs, and drugs related to treatment of neurodegenerative disorder, such as Alzheimer's disease. For example, the composition may further comprise a cholinesterase inhibitor, an N-methyl D-aspartate (NMDA) antagonist, an antidepressant, a gamma-secretase inhibitor, a beta-secretase inhibitor, an anti-Aβ antibody, an anti-tau antibody, an antagonist of the serotonin receptor 6, a p38alpha MAPK inhibitor, recombinant granulocyte macrophage colony-stimulating factor, a passive immunotherapy, an active vaccine, a tau protein aggregation inhibitor, an anti-inflammatory agent, a phosphodiesterase 9A inhibitor, a sigma-1 receptor agonist, a kinase inhibitor, a phosphatase activator, a phosphatase inhibitor, an angiotensin receptor blocker, a CB1 and/or CB2 endocannabinoid receptor partial agonist, a 3-2 adrenergic receptor agonist, a nicotinic acetylcholine receptor agonist, a 5-HT2A inverse agonist, an alpha-2c adrenergic receptor antagonist, a 5-HT 1A and 1D receptor agonist, a glutaminyl-peptide cyclotransferase inhibitor, a selective inhibitor of APP production, a monoamine oxidase B inhibitor, a glutamate receptor antagonist, an AMPA receptor agonist, a nerve growth factor stimulant, a HMG-CoA reductase inhibitor, a neurotrophic agent, a muscarinic M1 receptor agonist, a GABA receptor modulator, a PPAR-gamma agonist, a microtubule protein modulator, a calcium channel blocker, an antihypertensive agent, and a statin. Such therapies may be administered before, simultaneously, or following the administration of CAR expressing cells.

Any mammal may be treated by the methods described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In one example, the mammal is a human. The mammal can be any age or at any stage of development. The mammal may be male or female. In one instance, the mammal is an animal disease model.

In one example, the subject may be diagnosed with or as having a high risk of developing a neurodegenerative disease, or a disease associated with Aβ. A neurodegenerative disease, or a disease associated with Aβ may be Alzheimer's disease (AD), dementia, cerebral amyloid angiopathy (CAA) or amyloidosis.

A subject as described herein may be at risk of developing a neurodegenerative disease or a disease associated with Aβ. The subject may exhibit one or more clinical sign or symptom of a neurodegenerative disease or a disease associated with Aβ. The subject may be previously diagnosed with a neurodegenerative disease or a disease associated with Aβ. The subject may be amyloid positive. The subject may have dementia. The subject may have a clinical dementia rating (CDR) score of 0.5 to 1.0. The subject may have a CDR score of >1.0 to 2.0 (e.g., such as in moderate Alzheimer's disease). The subject may have a CDR score of >2.0.

The subject may or may not be symptomatic. Subjects with Alzheimer's disease may exhibit a variety of signs or symptoms, common signs or symptoms include loss of memory (e.g., short term memory or long-term memory), inhibition of reasoning capacity, inhibition or loss of ability to make decisions, impaired planning ability, changes to personality, and/or altered behavior patterns (e.g., depression, mood swings, loss of inhibition, apathy, and withdrawal). The symptoms of AD worsen over time, although the rate at which the disease progresses varies. On average, a subject with AD lives four to eight years after diagnosis, but can live as long as 20 years, depending on other factors. Changes in the brain related to AD begin years before any signs of the disease. This period, which can last for years, is referred to as preclinical AD. A subject in the early stage of AD (mild AD) may function independently, for example by driving, working and participating in social activities. A subject suffering from early AD may feel as if he or she is having memory lapses, such as forgetting familiar words or the location of everyday objects. Friends, family or others close to a subject with early AD may begin to notice difficulties. A doctor performing a detailed medical interview with a subject suffering from early AD may be able to detect problems in memory or concentration. A subject suffering from early-stage AD may experience one or more difficulties selected from a group consisting of: problems coming up with the right word or name, trouble remembering names when introduced to new people, challenges performing tasks in social or work settings, forgetting material that one has just read, losing or misplacing a valuable object, and increasing trouble with planning or organizing.

The middle stage of AD (moderate AD) may be the longest stage and may last for many years. As the disease progresses, a subject with AD may require a greater level of care. A subject suffering from the middle stage of AD may experience symptoms such as confusing words, getting frustrated or angry, and acting in unexpected ways (e.g., refusing to bathe or other personality changes). Neurodegeneration in the brain of a subject with moderate AD may make it difficult for the subject to express thoughts and perform routine tasks. Symptoms in a subject suffering from the middle stage of AD will be noticeable to others outside of close family. A subject suffering from the middle stage of AD experiences one or more difficulties such as forgetfulness of events or about the subject's own personal history, feeling moody or withdrawn, especially in socially or mentally challenging situations, being unable to recall the subject's own address or telephone number or the high school or college from which the subject graduated, confusion about where the subject is or what day it is, the need for help choosing proper clothing for the season or the occasion, trouble controlling bladder and bowels, changes in sleep patterns (e.g., sleeping during the day and becoming restless at night), an increased risk of wandering and becoming lost, personality and behavioral changes (e.g., suspiciousness and delusions) and compulsive, repetitive behavior (e.g., hand-wringing or tissue shredding).

In the final stage of AD (severe AD), a subject may lose the ability to respond to his or her environment, to carry on a conversation and, eventually, to control movement. A subject suffering from the final stage of AD may still say words or phrases, but communicating pain becomes difficult. A subject suffering from the final stage of AD may experience significant personality changes. A subject suffering from the final stage of AD may need extensive help with daily activities. A subject suffering from the final stage of AD may experience one or more difficulties such as requiring round-the-clock assistance with daily activities and personal care, losing awareness of recent experiences as well as of surroundings, experiencing changes in physical abilities (e.g., the ability to walk, sit and, eventually, swallow), having increasing difficulty communicating, and becoming vulnerable to infections (e.g., pneumonia).

The methods provided herein may be administered to a subject diagnosed with early stage, middle stage, or final stage of AD. The effect of treatment may be evaluated using the Alzheimer's Disease Assessment Scale-Cognitive Subscale (ADAS-Cog) and/or the AD Co-operative Study-Activities of Daily Living Inventory (ADCS-ADL). Alternatively, the effect of treatment on AD pathology in a subject may be evaluated by measuring the level of protein aggregates in the brain of the subject.

The methods described herein may encompass allogenic CAR therapy or autologous CAR therapy. The method may comprise adoptive transfer of CAR expressing cells. In such instances, the cells for example, macrophages or precursor cells of macrophages may be procured from the subject in need of the treatment, may be modified to express the CAR, and administered back into the subject. Alternatively, the cells for example, macrophages or precursor cells of macrophages may comprise cell lines or may be procured from donors. The procured cells then may be modified and administered into the subject.

The CAR expressing cell, may be administered in an effective dose. The effective dose may be either one or multiple doses, and are sufficient to produce the desired therapeutic effect, such as a reduction in the plaque load in the subject. An effective dose may comprise a dosage of of 1×10⁴ to 5×10⁹ cells/kg body weight, preferably 1×10⁴ to 2×10⁹ cells/kg body weight, including all integer values within those ranges. The optimal dosage and treatment regime for a particular subject can readily be determined by one skilled in the art of medicine by monitoring the subject for signs of disease and adjusting the treatment accordingly. Further, the effective dose may be calculated based on the stage of the disease and the health of the subject. In the situation where multiple doses are administered, that dose and the interval between the doses may be determined based on the subject's response to therapy.

### 7. Kits

Described herein is a kit comprising the vector or the cell expressing the CAR. The kit may further comprise instructions to perform the methods described herein.

The kit may include one or more containers comprising the vector, cells, and/or CAR described herein. The kit may further include a second therapeutic agent. The kit may further comprise suitable administration means like syringes, intravenous drip apparatus etc. Suitable containers include, for example, bottles, vials, syringes, assay plates, strips, matrices etc. The containers may be formed from a variety of materials such as glass, plastic, paper etc. The kit may further include other materials desirable from a commercial and user standpoint, including other buffers, diluents, filters, needles, and syringes.

The kit may further include a label or a package insert. The label or the package insert may comprise instructions for use and other information customarily included in commercial packages. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit. Said instructions may be derived from any of the methods as described herein

The kit may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like

The present invention has multiple aspects, illustrated by the following non-limiting examples.

### Example 1

### Aβ CAR-Ms Significantly Bind and Resorb Beta-Amyloid in Culture and Adopt a Unique CAR and Signaling-Induced Phenotype

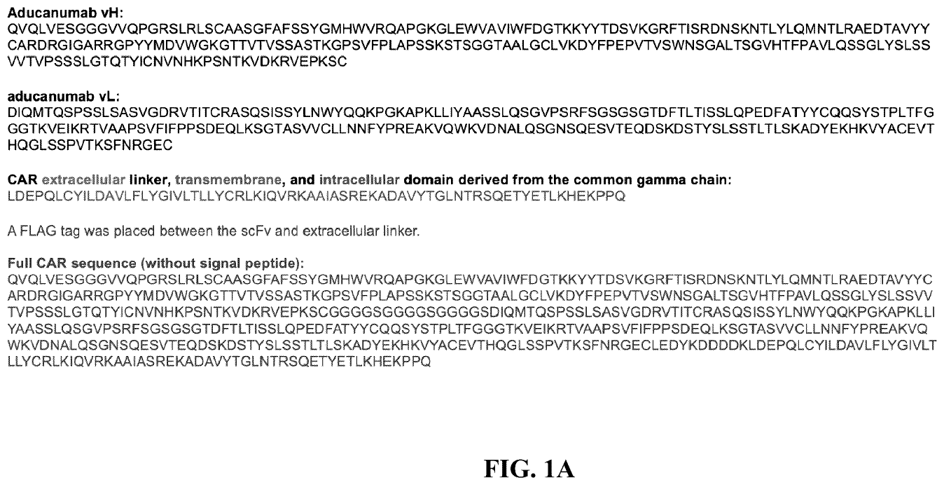

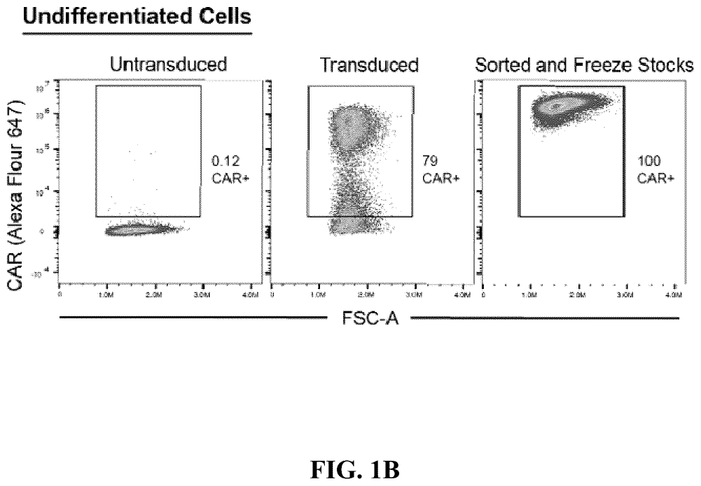

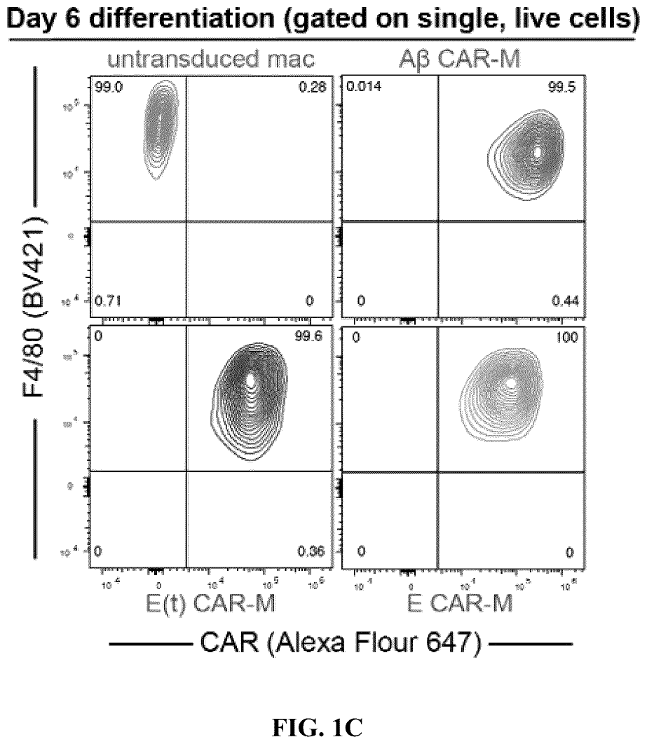

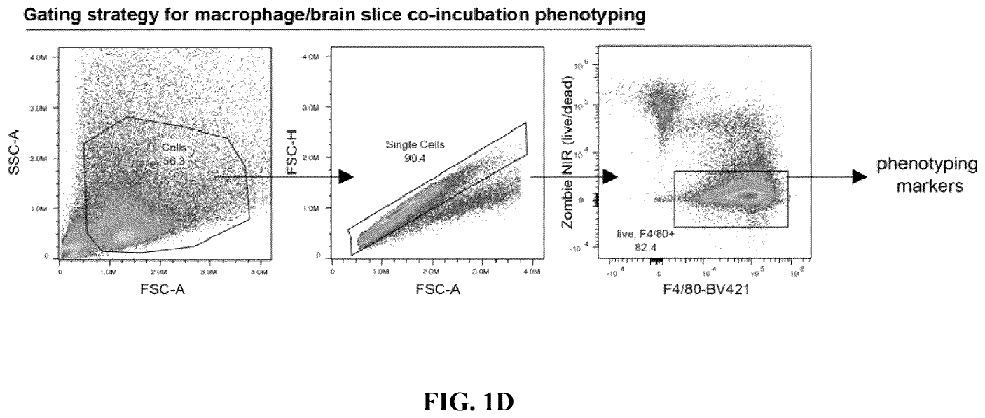

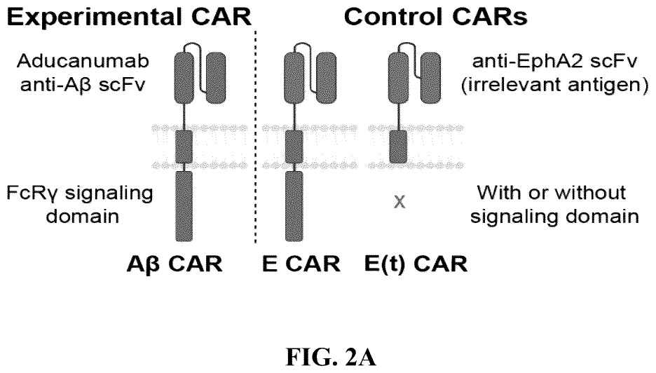

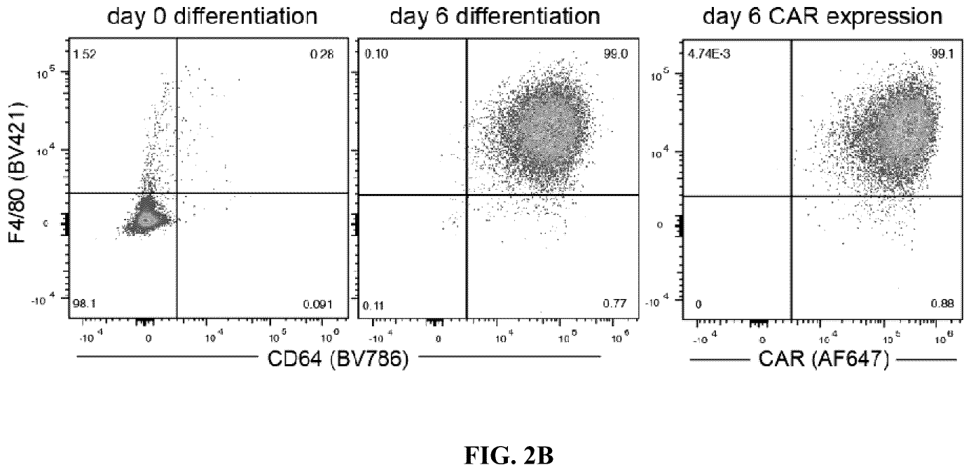

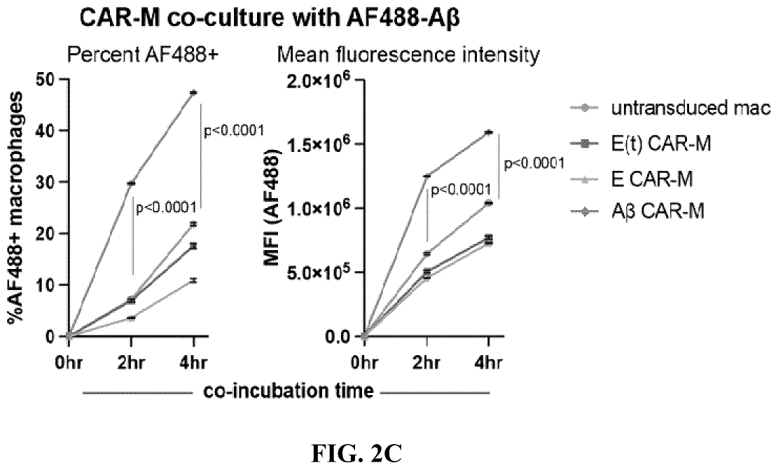

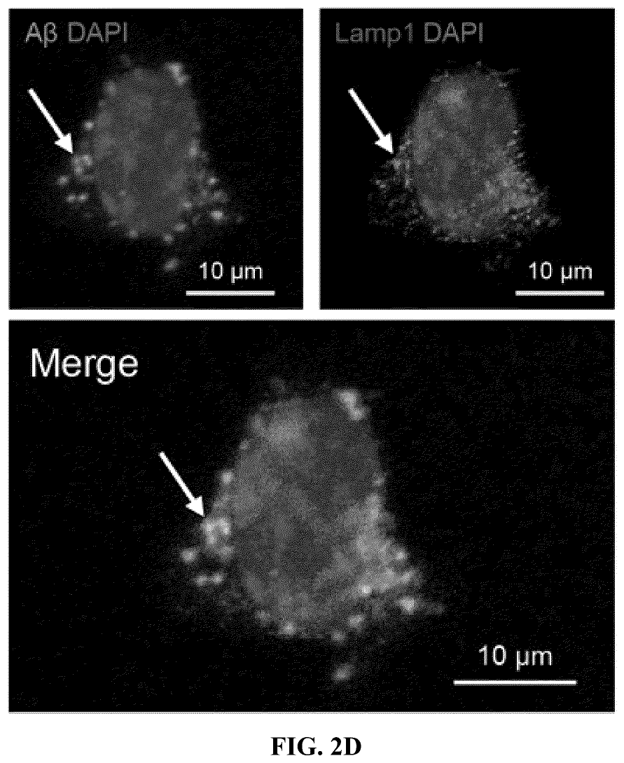

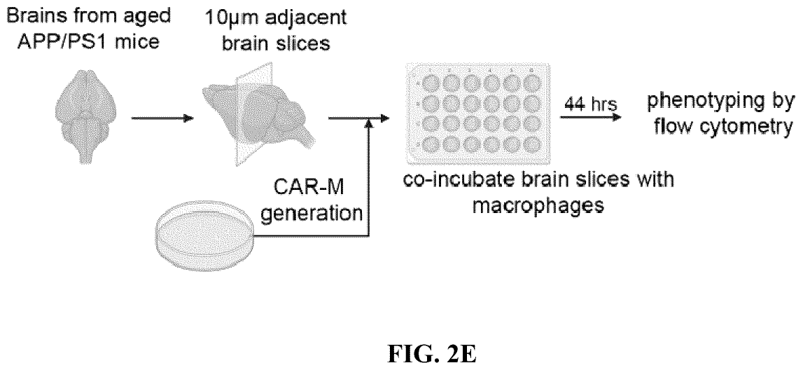

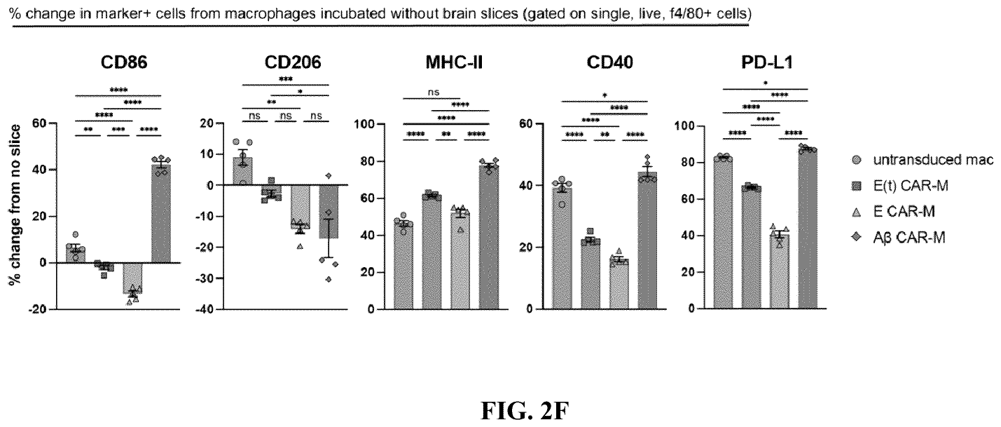

To reproducibly manufacture CAR-Ms that express the CAR to a high level and avoid the effects of variable transduction efficiency between experiments, control or experimental CAR constructs (FIG. 1B) were retrovirally introduced into the HoxB8 cell line, which was generated by transducing mouse bone marrow cells with the previously described estrogen-responsive HoxB8 construct that sustains the cells in an undifferentiated state in the presence of estrogen while maintaining their ability to differentiate into macrophages in the presence of M-CSF without estrogen. To discriminate between the effects of transduction with a CAR of similar structure but no target binding or signaling, as well as tonic signaling but no binding, two additional control CARs composed of 1) a non-specific extracellular scFv targeting EphA2 (not expressed on normal brain tissue with an intracellular FcRγ signaling domain, termed “E CAR-M” and 2) the same EphA2 scFv truncated after the transmembrane domain, thus containing no intracellular signaling domain, termed “E(t) CAR-M” (FIG. 2A) were generated.

First, surface CAR expression on macrophages after transduction and differentiation of HoxB8 cells in M-CSF (FIG. 2B) were confirmed. HoxB8 cells expressing control (E CAR-M and E(t) CAR-M) or Aβ-targeting CARs to a matched level were sorted and stocks were generated to ensure consistent CAR expression in all experiments (FIG. 1C). To test CAR function, untransduced macrophages, E CAR-Ms, E(t) CAR-Ms, or Aβ CAR-Ms were co-cultured with AF-488-labeled Aβ (1-42) and analyzed by flow cytometry, which demonstrated a significant time and Aβ CAR-dependent increase in the percentage of cells that took up Aβ as well as the amount of Aβ taken up by each cell (FIG. 2C). Aβ uptake into Aβ CAR-Ms was confirmed using confocal microscopy, which localized Aβ to punctate intracellular vesicles and the Lamp1 expressing lysosomal compartment (FIG. 2D).

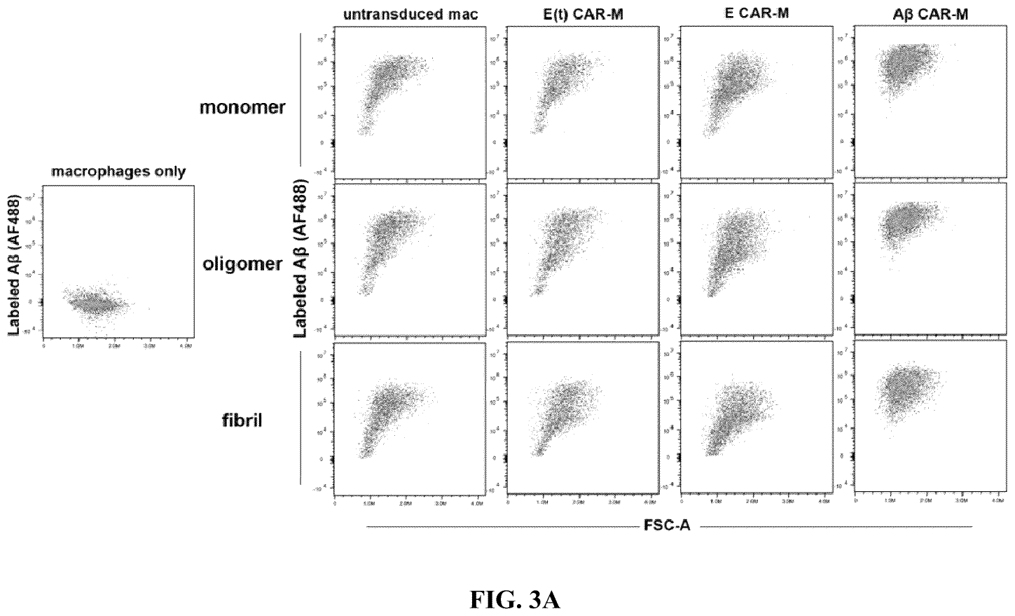

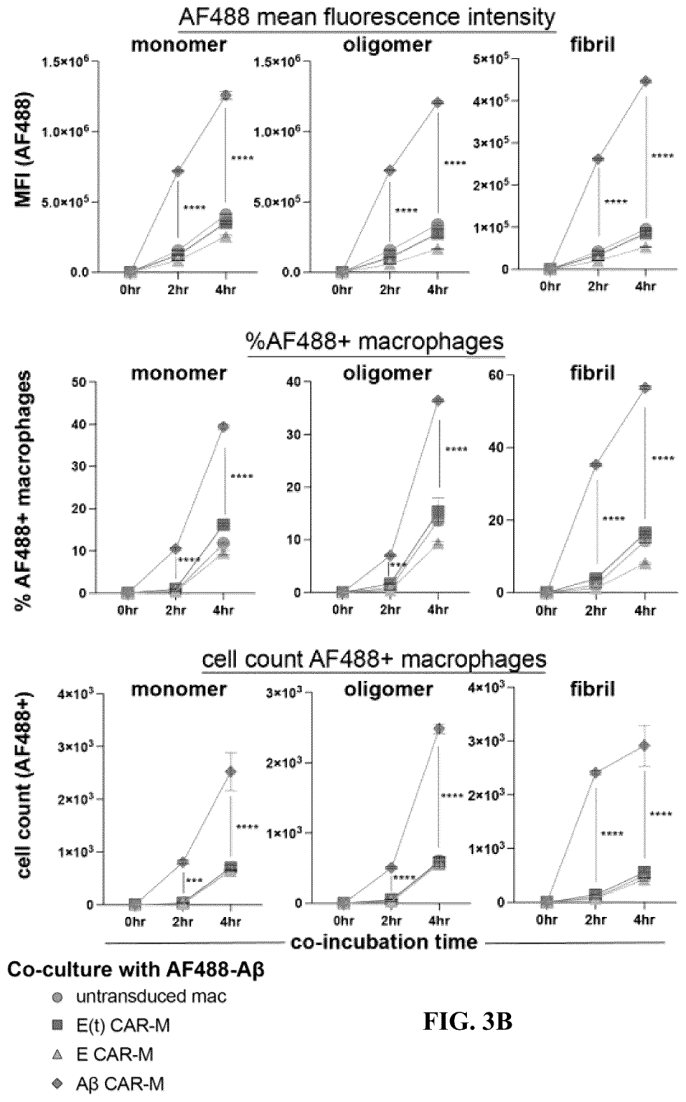

The Aβ CAR construct contains an extracellular binding domain derived from the aducanumab antibody, which is known to preferentially bind aggregated forms of Aβ, including oligomers and fibrils. Thus, it was further tested whether Aβ CAR-Ms could also take up Aβ oligomers and fibrils. To measure Aβ uptake, monomeric, oligomeric, or fibril forms of Aβ were labeled with a fluorescent protein, AF488, and co-cultured controls or Aβ CAR-Ms with these three forms of Aβ for 2 or 4 hours. Aβ uptake was assessed by measuring AF488 fluorescence in the macrophages by flow cytometry, which confirmed that Aβ CAR-Ms more effectively took up all three forms of Aβ compared to untransduced or control CAR macrophages (FIGS. 3A-3B).

To assess the effect of CAR signaling on macrophage phenotype using a more physiologically relevant target, control and CAR-Ms were cultured on amyloid laden brain slices from aged APP/PS1 mice. Then, downstream functional molecules putatively modulated by Fc receptor signaling were examined, before and after exposure to Aβ-containing brain slices using flow cytometry (FIG. 1D, FIGS. 2E-2F). Compared to controls, Aβ CAR-Ms upregulated CD86, MHC-II, CD40, and PD-L1 and downregulated CD206 after co-incubation with amyloid-laden brain slices. An effect of transduction alone is observed by comparing untransduced macrophages to E(t) CAR-Ms, and an effect of tonic signaling alone is seen by comparing E(t) CAR-Ms versus E CAR-Ms. These in vitro experiments demonstrate that the CAR-Ms undergo Aβ CAR-dependent, target-induced phenotype changes.

Because the CAR exerts effects through both target binding and intracellular signaling, the rest of the studies were continued with experimental Aβ CAR-Ms and control E(t) CAR-Ms, which will be henceforth referred to as “control CAR-M.”

### Example 2

### Aβ CAR-Ms Significantly Reduce Plaque Load on Brain Slices Ex Vivo, and Resorb Plaques of all Sizes

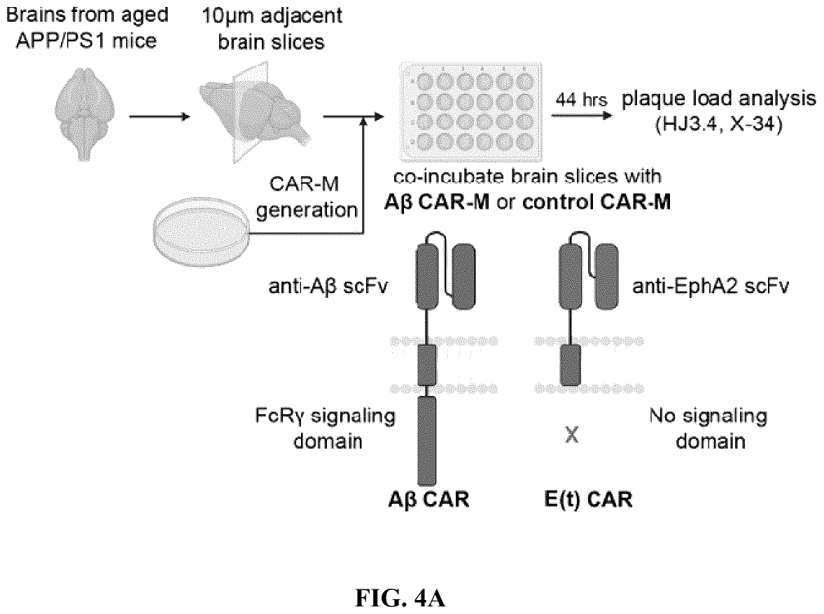

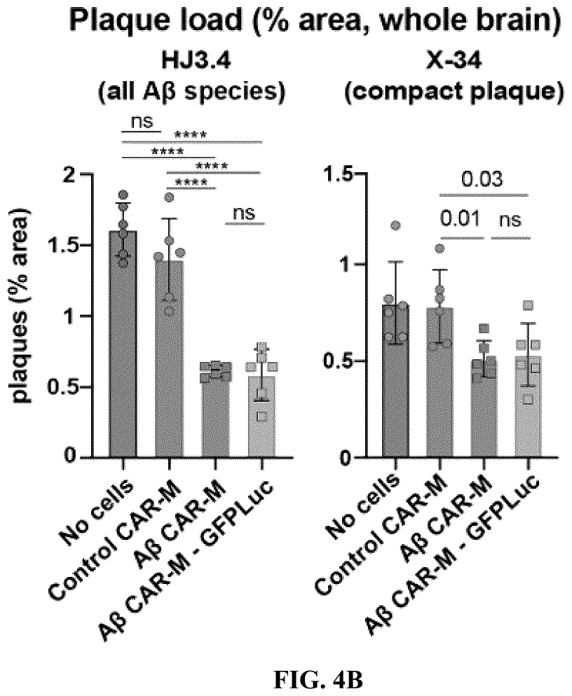

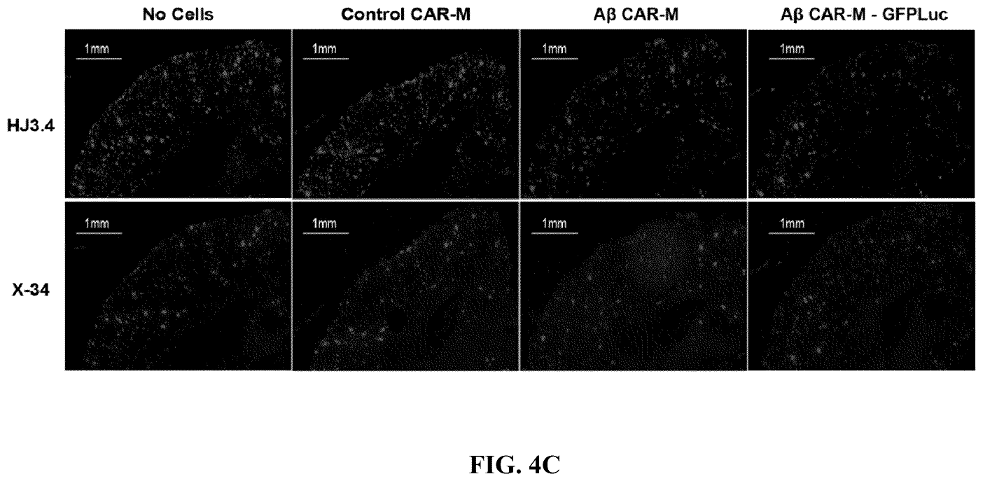

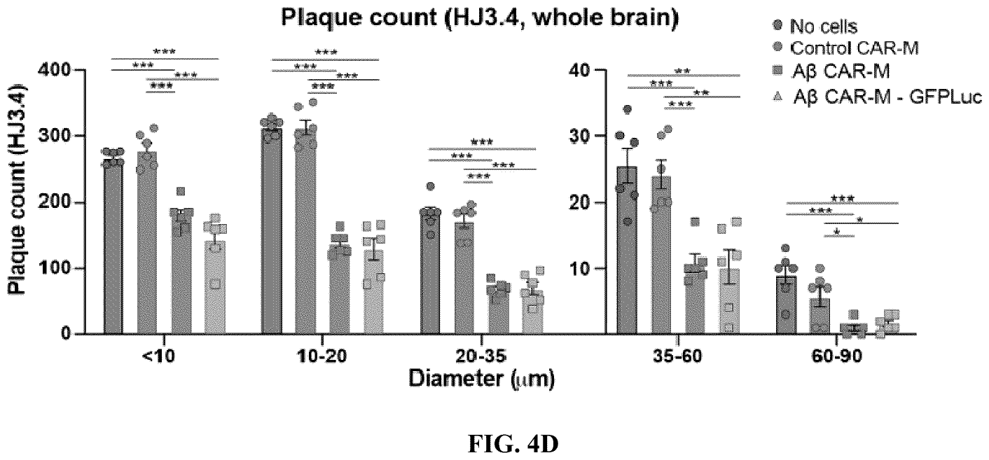

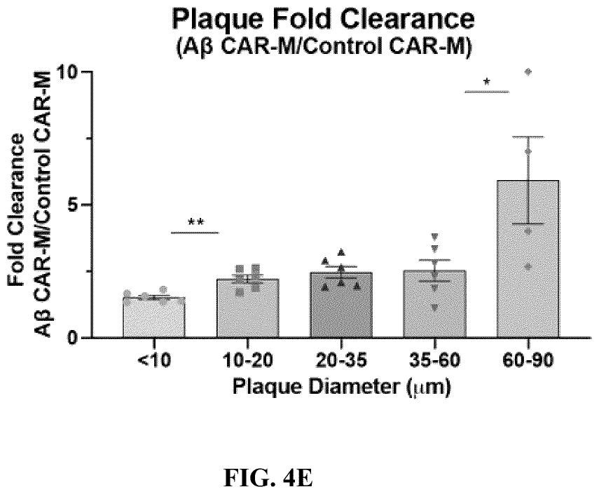

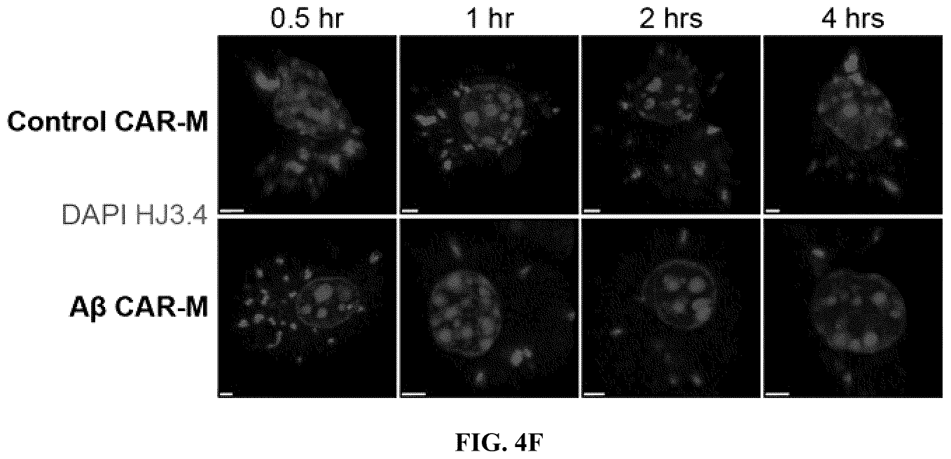

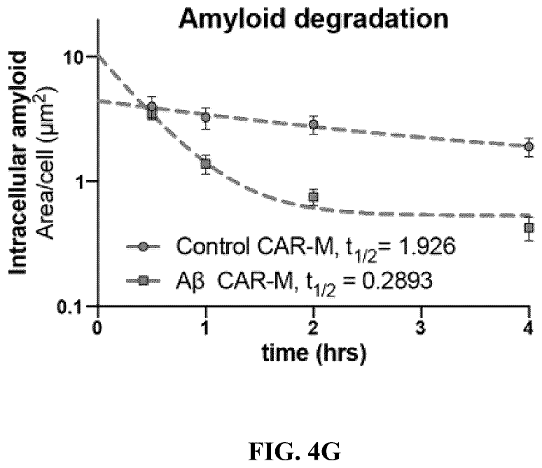

To assess the ability of Aβ CAR-Ms to resorb plaques that were deposited in vivo, control or Aβ CAR-Ms were co-incubated on brain slices from aged APP/PS1 mice (FIG. 4A), and this time also introduced GFP-Luciferase (GFP-Luc) into the cells in preparation for in vivo monitoring. Quantification of HJ3.4 and X-34-stained plaques, which recognizes all forms of Aβ and fibrillar compact plaques, respectively, revealed a significant reduction in plaque burden in Aβ CAR-M treated slices over control CAR-M slices ex vivo, with no detrimental effect of additional GFP-Luc expression (FIGS. 4B-4C). Thus, all subsequent studies used GFP-Luc expressing control and Aβ CAR-Ms.

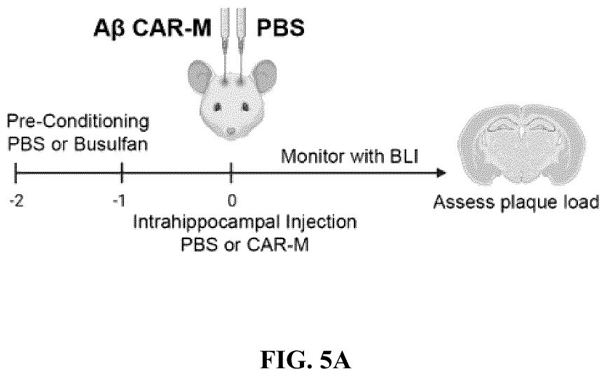

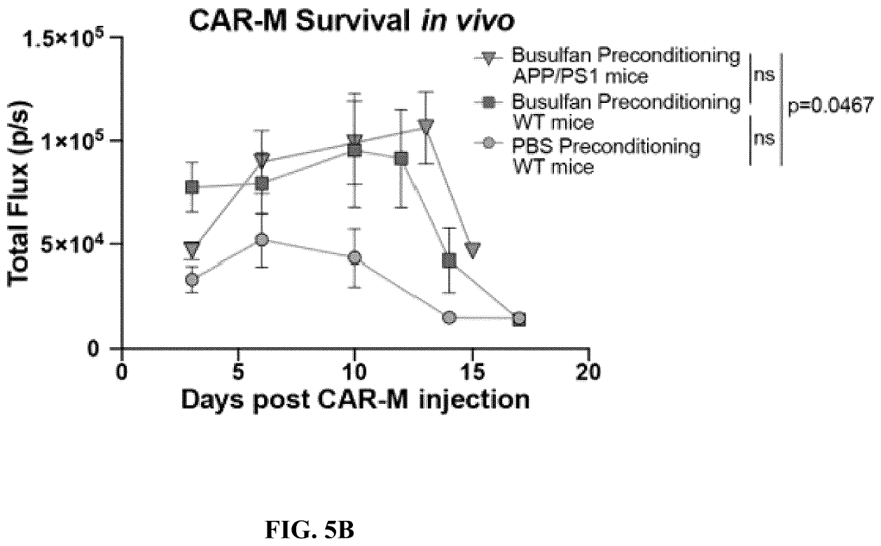

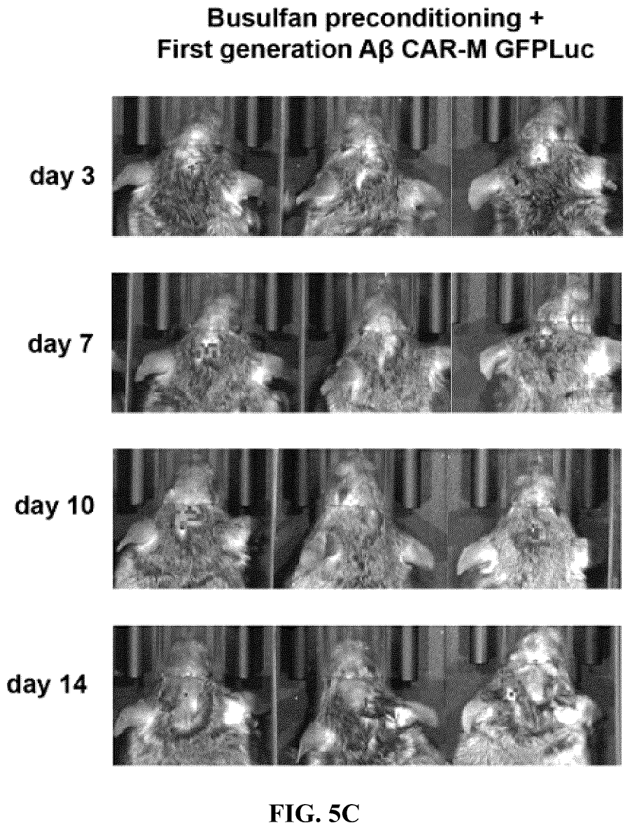

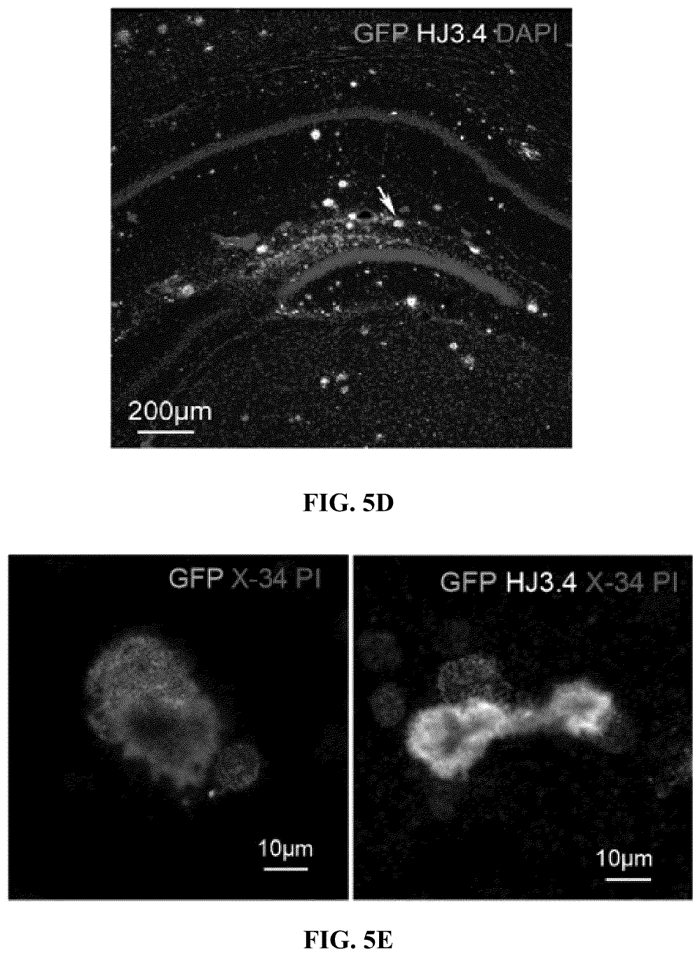

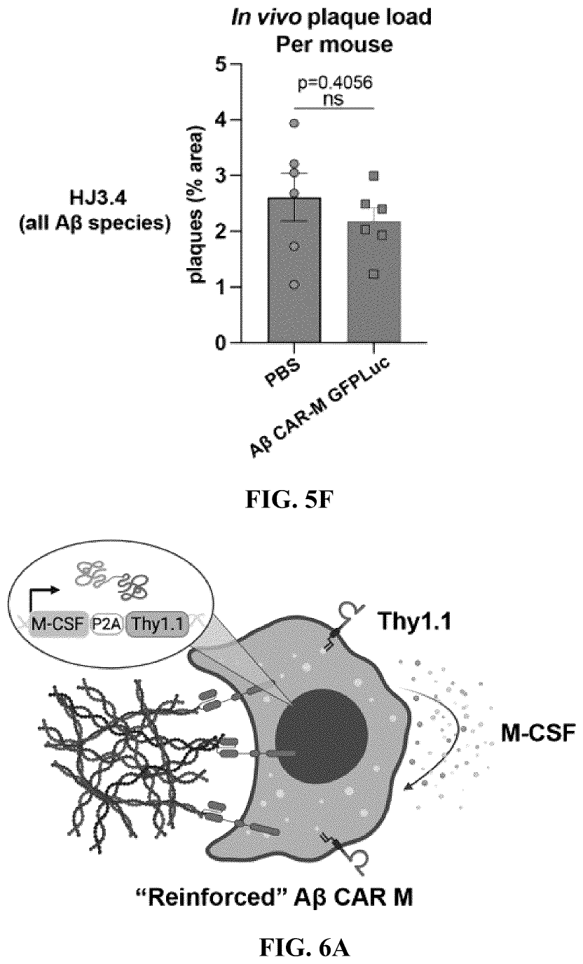

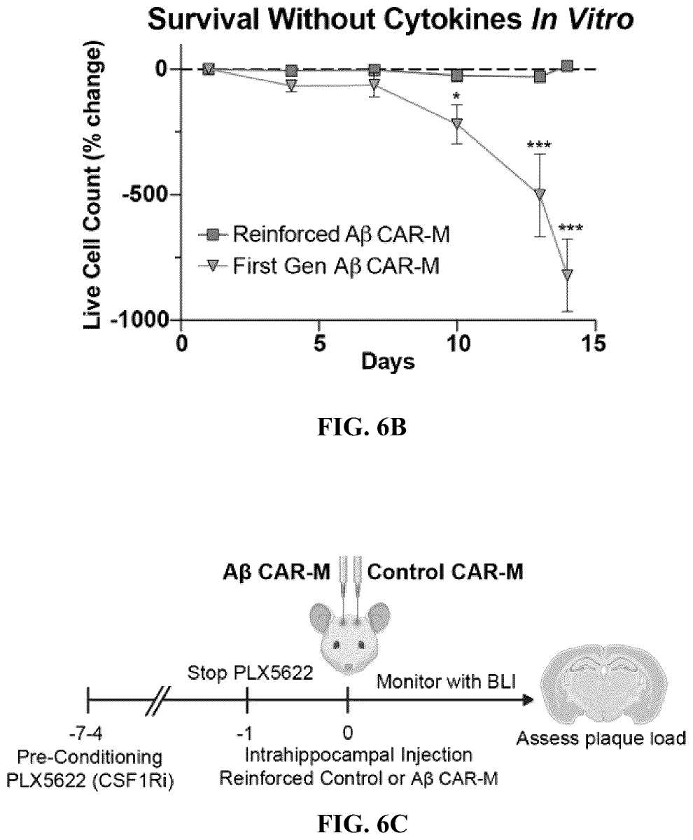

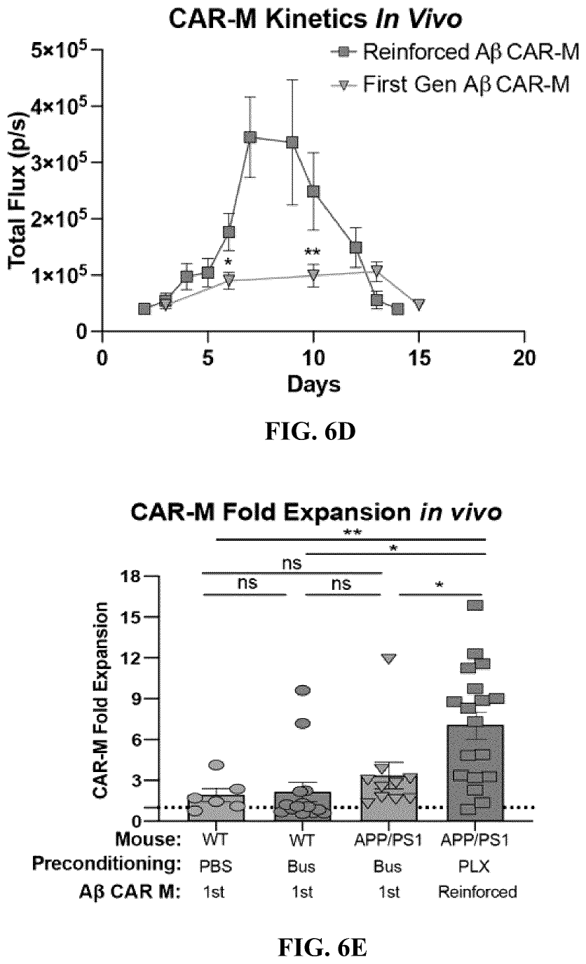

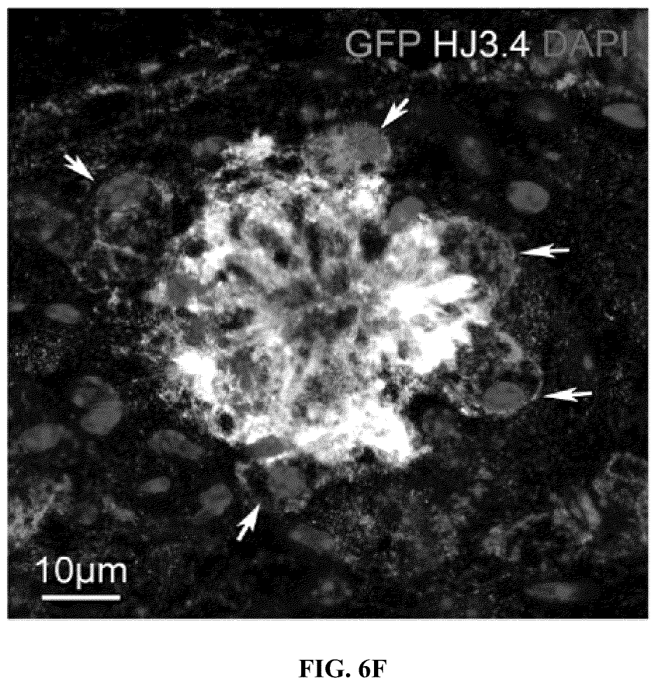

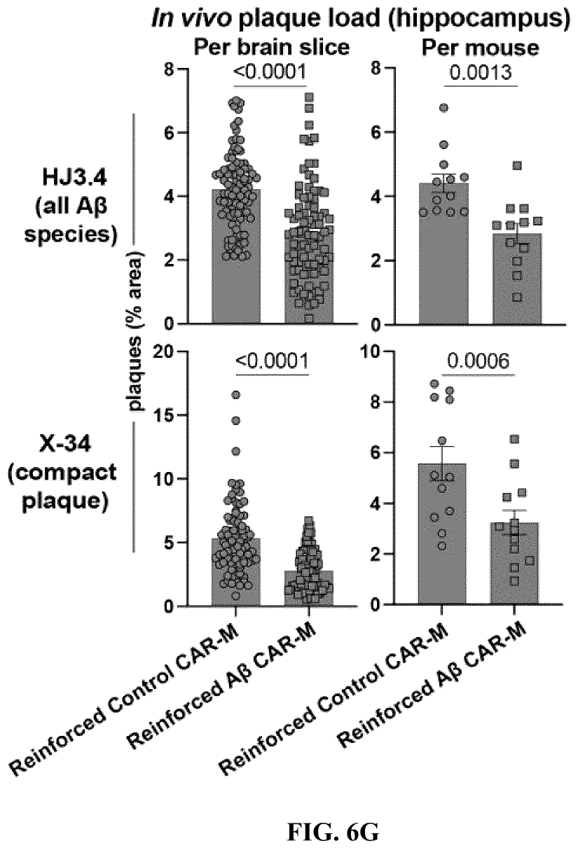

Further analysis of the sizes of plaques being resorbed on APP/PS1 brain slices showed that Aβ CAR-Ms effectively resorb plaques of all sizes (FIG. 4D). In addition, when fold change in plaque clearance of Aβ-targeted versus control CAR-Ms was assessed, targeted CAR-Ms resorbed larger plaques of >10 microns relatively more effectively than smaller plaques (FIG. 4E). This, as well as direct immunohistological visualization of plaque-CAR-M interactions (FIG. 5E, FIG. 6F), suggests that plaque is likely resorbed in pieces rather than being taken up by whole plaque phagocytosis.

### Example 3

### Aβ CAR-Ms Degrade Intracellular Amyloid

In addition to taking up Aβ, it was determined if Aβ CAR-Ms could more efficiently degrade the resorbed intracellular Aβ. CAR-Ms were incubated with amyloid-laden brain slices for 4 hours; cells were then removed from the slices and grown in culture. At various timepoints thereafter, cells were fixed and immunostained with HJ3.4 to track intracellular amyloid over time (FIG. 4F). The area of Aβ immunostaining per cell was quantified and plotted it over time. Intriguingly, although Aβ CAR-Ms take up more Aβ, they also degrade it more efficiently compared to control CAR-Ms (FIG. 4G). The half-life of intracellular amyloid in control CAR-Ms was 1.926 hours, while the half-life in Aβ CAR-Ms was only 0.2893 hours. Thus, Aβ CAR-Ms not only take up more Aβ than control CAR-Ms, but also degrade intracellular Aβ faster.

### Example 4

First-Generation Aβ CAR-Ms Find Plaque In Vivo but have Limited Expansion and Survival

Next, the ability of Aβ CAR-Ms to target and reduce amyloid plaques in vivo were tested. Given the importance of preconditioning in CAR T-cell therapy and microglia engraftment studies, mice were preconditioned with a non-ablative dose of Busulfan for two days to deplete endogenous microglia prior to intrahippocampal injection of PBS or Aβ CAR-Ms (FIG. 5A). As it was uncertain if the CAR-Ms would spread within the brain, we chose to inject one hippocampus with cells and the contralateral hippocampus with PBS (vehicle).

Engraftment of CAR-Ms was assessed using bioluminescence imaging (BLI) in WT and APP/PS1 mice, which showed that in APP/PS1 mice, CAR-Ms survived and modestly expanded for the first 10-12 days, but subsequently diminished to baseline by day 14 (FIG. 5B-5C). Plaque load was quantified on day 14 in a circular region of interest (ROI) surrounding the injection site, centered around residual GFP signal in the hippocampal region (FIG. 5D). Uniform circle sizes were used across all slices. Aβ CAR-Ms recognized and bound Aβ plaques (FIG. 5E); however, no significant difference in plaque load was observed between PBS and Aβ CAR-M treated hemispheres (FIG. 5F). Because the injected CAR-Ms appeared to stay relatively localized to the injection site (FIG. 5D), for subsequent studies decision was made to inject the left hippocampus of each mouse with control CAR-Ms and the right hippocampus with Aβ CAR-Ms. This bilateral injection paradigm also controls for inter-mouse heterogeneity in plaque load.

### Example 5

“Reinforced CAR-Ms” Expressing M-CSF have Enhanced Survival and Locally Reduce Hippocampal Plaque Load In Vivo

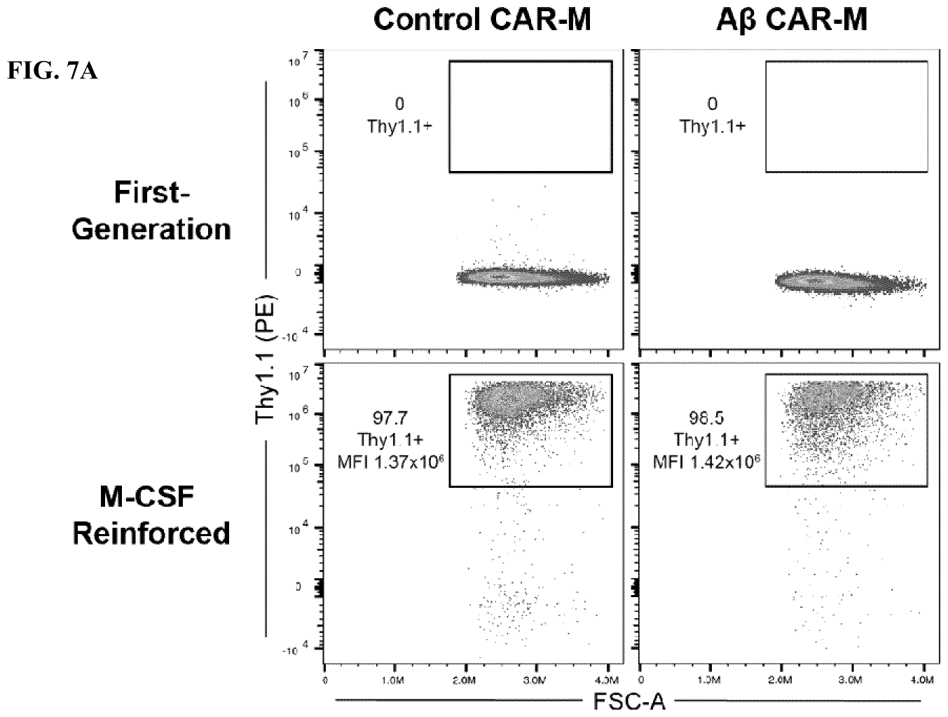

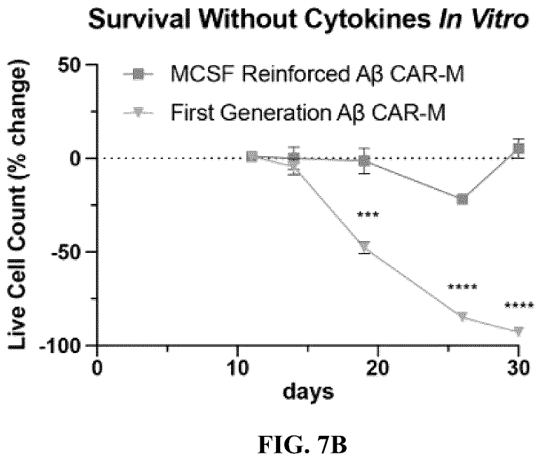

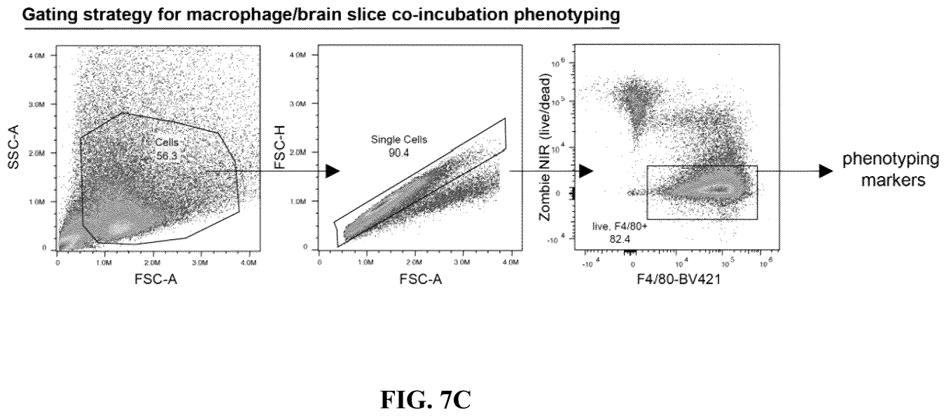

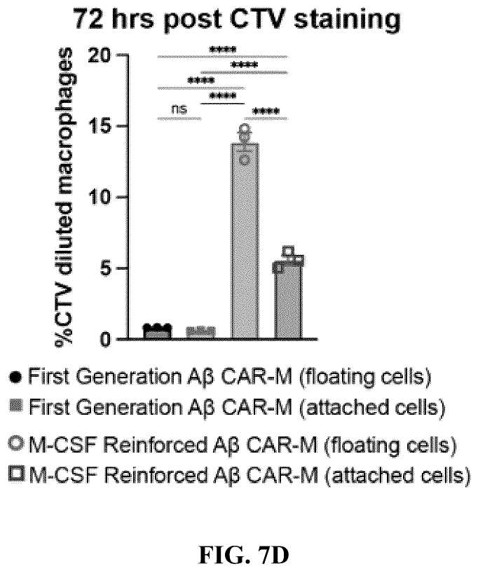

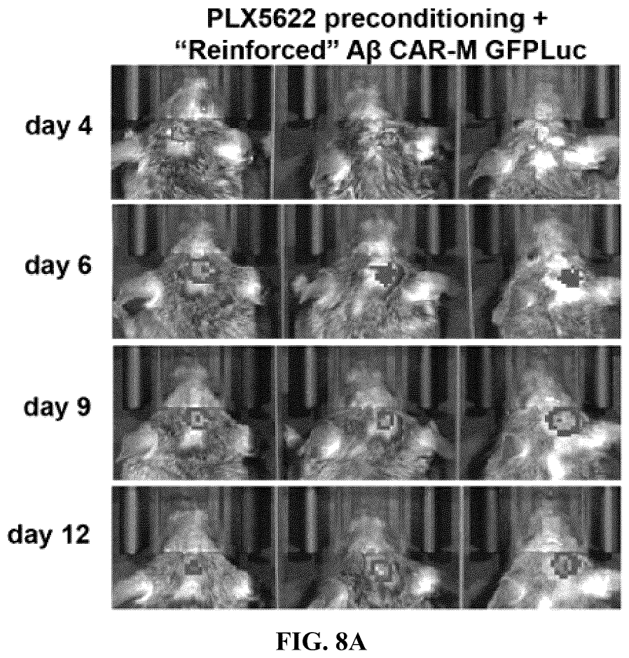

Due to the lack of survival, persistence, and efficacy of first-generation CAR-Ms in vivo, “reinforced CAR-Ms” were developed that secret M-CSF- to improve persistence and efficacy in vivo (FIG. 6A, FIG. 7A). While first-generation CAR-Ms nearly completely die within two weeks without exogenous cytokine, M-CSF reinforced CAR-Ms persist at a stable level in vitro in the absence of exogenous cytokine for at least a month (FIG. 6B, FIG. 7B).

Reinforced Aβ CAR-Ms express F4/80, CD64, and CCR2, and lack expression of Ly6C and CD62L, suggesting they become mature macrophages after M-CSF differentiation (FIG. 7C). To further characterize these cells, M-CSF reinforced and first-generation Aβ CAR-Ms were stained with Cell Trace Violet (CTV) to measure their proliferation in vitro in the absence of exogenous M-CSF. A small proliferating population within the floating cells, and a larger non-proliferating population of attached cells exist in both cultures. However, in M-CSF reinforced CAR-M's, there is a significantly larger proliferating population (FIG. 7D). For all experimental studies, we only use attached cells with less proliferative capacity but more homogenous differentiation.

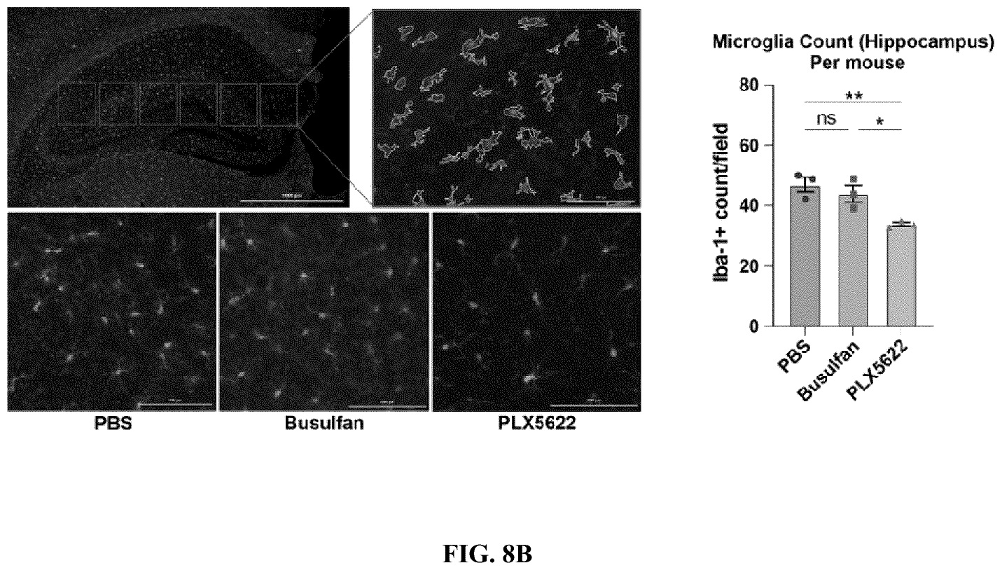

To assess in vivo survival and expansion of reinforced CAR-Ms, we injected M-CSF reinforced control and Aβ CAR-Ms in opposite hippocampi of aged APP/PS1 mice. To deplete endogenous microglia while sparing non-myeloid cells with a potentially more clinically translatable drug, mice were subjected to four to seven days of preconditioning with the CSF-1 inhibitor PLX5622 (FIG. 6C). BLI showed M-CSF reinforced CAR-Ms rapidly expanded in the first week within the brain, then plateaued and rapidly contracted (FIG. 6D, FIG. 8A). Although only a modest microglial depletion was observed with this preconditioning regimen compared with PBS or low-dose Busulfan preconditioning (FIG. 8B), reinforced CAR-Ms reached significantly greater numbers than first-generation CAR-Ms (FIG. 6D), expanding in all but one of 18 mice with an average 7-fold expansion in vivo (FIG. 6E).

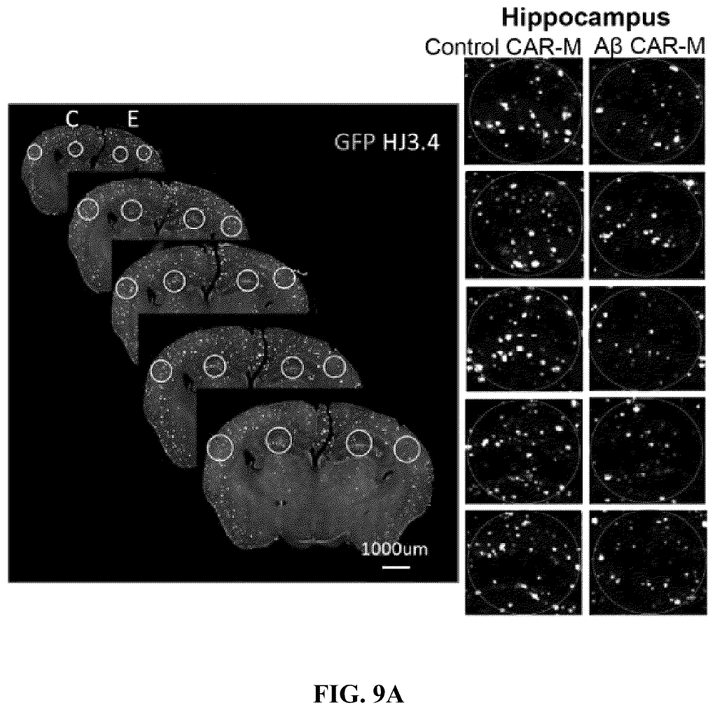

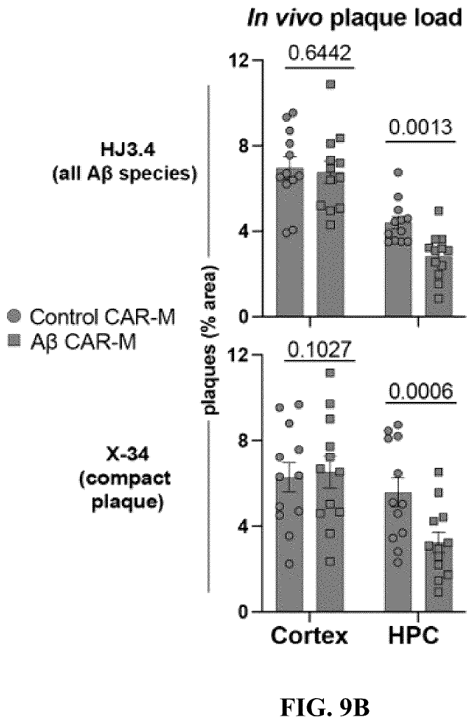

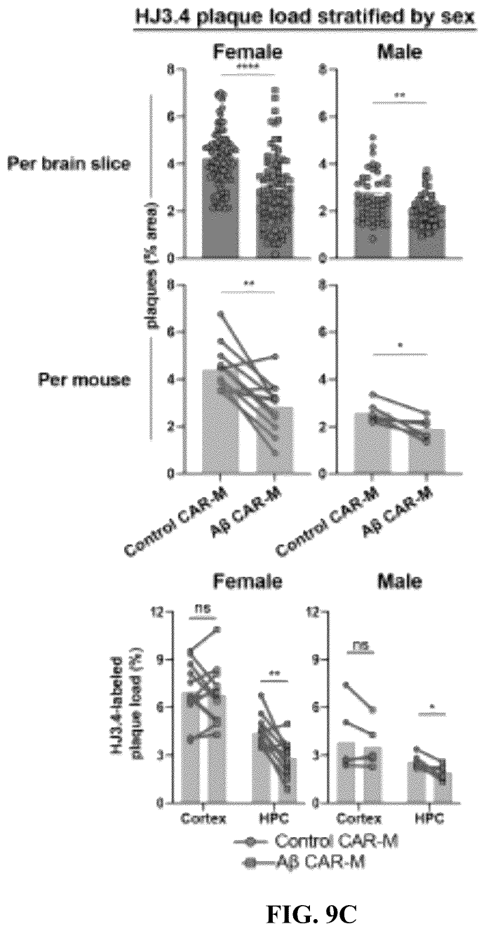

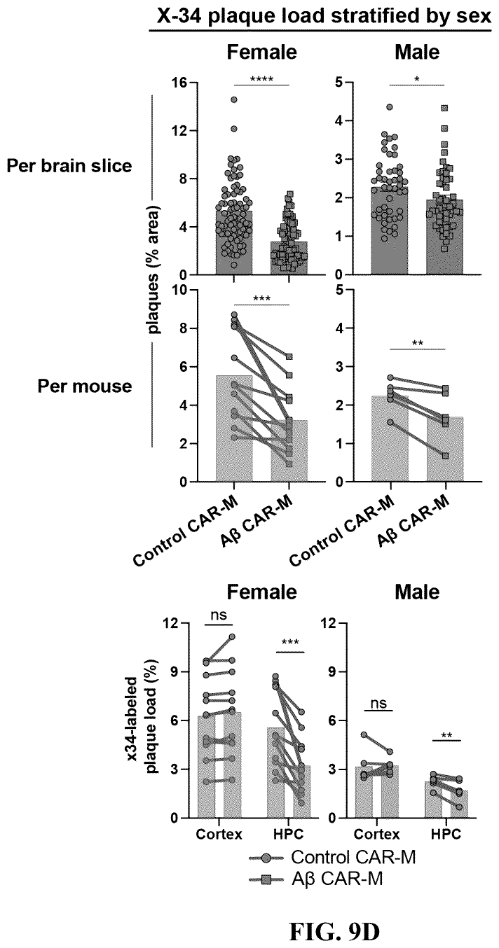

Histologic analysis demonstrated that Aβ CAR-Ms recognized plaque in vivo and demonstrated plaque phagocytosis (FIG. 6F). Quantification of plaque load in the previously defined ROI around the injection site (FIG. 9A) revealed a significant reduction in diffuse and compact plaques after M-CSF reinforced Aβ CAR-M treatment compared to M-CSF reinforced control CAR-M treatment. This result holds whether plaque load is analyzed per brain slice or per individual mouse (FIG. 6G). A little migration of CAR-Ms away from the injection site was again observed, and analysis of random ROIs in mirror regions of the cortex did not reveal significant changes in plaque load (FIG. 9A-9B), demonstrating relative uniformity of plaque load across hemispheres and no change in plaque load in regions CAR-Ms were not present. These results hold when these data are stratified by sex (FIG. 9C-9D).

### SUMMARY

In the study disclosed in the examples, it was shown that macrophages expressing an Aβ-targeting CAR take up Aβ peptide in vitro and significantly reduce plaque load on amyloid-laden brain slices ex vivo. It was further shown that M-CSF reinforced Aβ CAR-Ms significantly reduce diffuse and compact hippocampal plaque load in vivo when injected into the hippocampus of aged APP/PS1 mice after preconditioning with a CSF-1 inhibitor to deplete endogenous microglia.

These results demonstrated that while macrophages expressing a CAR targeting Aβ can significantly resorb Aβ and amyloid plaques in vitro and ex vivo, achieving significant in vivo plaque resorption, but further required engineering the cells to self-sustain by producing M-CSF for autocrine and/or paracrine stimulation. While M-CSF secretion alone may have achieved some degree of plaque clearance through either microglia stimulation or improving CSF flow dynamics, a significant independent CAR target-mediated effect was measured, since the control hemispheres were also treated with M-CSF secreting CAR-Ms but not targeting A3. It was found that Aβ CAR signaling also significantly changes the phenotype of macrophages upon target binding in vitro, which may itself affect both how the CAR-M functions and how it modulates the local microenvironment. Since engineering M-CSF secretion into the CAR-M had a significant effect, creating additional “Reinforced CAR-Ms” that secrete other microenvironment-modulating cytokines of interest may be another future approach to rationally reshape the local environment.

CAR-Ms injected into the hippocampus of APP/PS1 mice were limited in their survival and migration within the brain niche. It was not examined why the CAR-Ms fail to persist long-term within the brain, but several possibilities exist. Just as microglia have different phenotypes throughout the brain due to differences in local microenvironment, fully differentiated CAR-Ms may lack the complete set of receptors necessary to thrive within the brain niche. Even if they can survive within the brain, they may become out-competed by more fit microglia as they recover from preconditioning depletion. Alternatively, the CAR-Ms may be immunologically depleted, due to either MHC expression of foreign antigenic peptides (such as from GFP-Luciferase, or due to mixed strain immunoreactivity (APP/PS1 mice are on a mixed C57BL/6×C3H background, since pure C57BL/6 APP/PS1 mice develop seizures at a considerable rate). While the mechanism of failure of long-term CAR-M engraftment into the brain is of interest academically, it is not clear whether achieving long-term engraftment would be desired clinically. The fact that these CAR-Ms are allogeneic and do not persist long-term, but still have a significant local effect in vivo is encouraging for the possibility that if CAR-Ms were to become therapeutically relevant for AD in the future, an allogeneic product may be feasible. Next step studies that improve migration throughout the brain, and ideally CNS penetration after IV delivery, will be important for achieving full clinical potential. The previous work showed that peripheral monocytes can enter the AD brain in APP/PS1 mice and reduce plaque load in the absence of any intervention, suggesting that increasing the number of peripheral monocytes entering the brain, especially Aβ CAR-modified ones, will further reduce plaque load. Enhancing CAR-M's with factors that promote peripheral myeloid cell entry, adaptation, and migration to the brain niche, may be additional strategies to boost therapeutic potential. Another potential strategy for clinical translation is the delivery of anti-A CAR mRNA with monocyte/macrophage-targeted lipid nanoparticles, as has been done for CAR-T cells in the setting of cardiac fibrosis.

In several neurological diseases, including AD, the microglial niche is known to undergo phenotypic changes that can be both beneficial and detrimental to disease progression. Because of these insights, there have been several efforts to replace dysfunctional microglia with healthy microglia or with peripheral monocytes via microglial or bone marrow transplant after preconditioning with myeloablative chemotherapy or radiation to remove endogenous microglia. These studies also showed that while monocytes do not infiltrate the brain parenchyma under homeostatic conditions, monocytes can infiltrate the brain parenchyma in the setting of preconditioning chemotherapy or radiation, which in addition to depleting endogenous microglia to make room for new cells to engraft, presumably promotes peripheral monocyte infiltration into the brain due to disruption of entry sites into the central nervous system, including the blood-brain-barrier, and increased secretion of cytokines and chemokines that recruit peripheral immune cells. In line with these studies, it was also observed that preconditioning with agents that reduce endogenous microglia, in our case non-myeloablative (low dose) Busulfan, improves engraftment of peripheral myeloid cells in both WT and APP/PS1 mice compared to PBS preconditioning. Better microglial removal has been achieved with myeloablative doses of Busulfan followed by bone marrow transplant; however, we intentionally did not pursue fully ablative regimens that have little potential for clinical translation. PLX5622 is reported to robustly deplete microglia as well as all other myeloid cells in other models if given ad libitum in the chow for an extended period. In the disclosed studies, this more specific microglia-depleting preconditioning regimen was associated with significantly reduced plaque load in the hippocampus of Aβ CAR-M treated APP/PS1 mice. Since a similar PLX CSF1R inhibitor is currently FDA approved for a rare myeloid-derived tumor and has been well tolerated, this may be a more clinically translatable preconditioning regimen. A modest microglia reduction after delivering the drug intra-peritoneally twice a day for four days was observed, and although microglia are expected to quickly recover after discontinuation of PLX5622, it was still observed substantial proliferation of M-CSF reinforced CAR-Ms in that setting.

Accumulating evidence suggests that peripheral monocytes/macrophages phagocytose and degrade Aβ plaques more effectively than microglia. Transgenic mice that block peripheral myeloid cell infiltration into the brain parenchyma have increased Aβ plaque load compared to control APP/PS1 mice, suggesting that peripherally derived myeloid cells phagocytose and eliminate plaques better than brain-resident microglia. Conversely, the depletion of microglia in APP/PS1 mice led to no change in amyloid plaque count or size that developed over time, again implying that microglia are unable to effectively phagocytose and degrade amyloid plaques, though the ability of peripheral macrophages to reduce amyloid plaque load was not assessed in this study. The inability of late stage disease microglia to control plaque load with AD progression may be attributed to the reduced expression of Aβ binding receptors and Aβ-degrading enzymes as plaque deposition increases with age, which was shown to lead to decreased phagocytosis and degradation of amyloid material in APP/PS1 mice(50). Similarly, microglia isolated from human AD brains show reduced expression of molecules important for phagocytosis and the recycling of phagocytic receptors. Recent reports suggest that microglia may even promote the spread and development of Aβ plaques. Peripheral macrophages, however, may also be defective at phagocytosing Aβ plaques in the setting of AD potentially due to chronic Aβ exposure, as it has been shown that monocytes/macrophages from AD patients were less effective at phagocytosing Aβ compared to cells from age-matched non-AD patients. Allogenic CAR-M therapy may be a solution to overcome dysfunctional cell-mediated amyloid plaque clearance in AD and provides a theoretical advantage over therapeutic strategies that require functional endogenous cells to remove Aβ.

While a direct comparison of Aβ CAR-Ms and anti-Aβ monoclonal antibodies was not conducted, theoretical advantages of CAR-Ms include their ability to be engineered to express factors that can reshape or reinforce cell phenotype or local environments, and constitutive phagocytosis and degradation of plaque material due to endogenous CAR expression that does not rely on antibody encounter or resident microglia for Fc mediated uptake. Further, while endogenous microglia or macrophages may become dysfunctional with age, CAR-Ms could theoretically be engineered from allogeneic young healthy donors to overcome functional challenges facing aged cells.

The findings that Aβ CAR-Ms not only take up more amyloid, but also degrade it more quickly and to a greater degree than control macrophages implies that the CAR enhances the resorptive capcity of the cell as well as intracellular processing of resorbed contents. There may be additional functional consequences resulting from retained intracellular amyloid, which remain to be fully examined. Mechanistically, FcRγ signaling has been shown to drive the expression of lysosomal genes involved in degradation and killing of lysosomal cargo, suggesting that additional FcRγ signaling from the intracellular signaling domain upon amyloid binding in Aβ CAR-Ms may explain the increased rate and magnitude of amyloid degradation observed. FcRγ signaling can activate lysosomal gene expression changes through the transcription factor TFEB, which is also known to enhance Aβ degradation in vivo through lysosomal biogenesis and degradative functions.

The ability of CAR-Ms to degrade amyloid rather than redistribute it may also lead to reduced incidence of amyloid-related imaging abnormalities (ARIA) compared to antibody therapy. ARIA is thought to occur in the setting of anti-Aβ monoclonal antibody therapy when amyloid-antibody complexes are cleared via perivascular clearance mechanisms and accumulate in perivascular spaces, which can impair further perivascular drainage of these complexes and cause inflammatory reactions that damage the brain vasculature and disrupt the blood-brain-barrier, resulting in edema and microhemorrhage. Future studies will determine if CAR-Ms can reduce amyloid plaque burden with less incidence of ARIA than anti-Aβ monoclonal antibody therapy.

Although Aβ were used as the target of these CAR-Ms, theoretically any pathogenic material may be targeted and degraded by replacing the scFv domain on this CAR backbone. These data support the further development of CAR-M technology beyond cancer, and establish CAR-Ms as one additional potential approach in the therapeutic toolbox for AD.

In summary, genetically engineering macrophages to express an Aβ-targeting chimeric antigen receptor (CAR-Ms) was developed to target and degrade amyloid plaques. When injected intrahippocampally, first-generation CAR-Ms have limited persistence and fail to significantly reduce plaque load, which led to engineering a next-generation CAR-Ms that secrete M-CSF and self-maintain without exogenous cytokines. Cytokine secreting “reinforced CAR-Ms” have greater survival in the brain niche, and significantly reduce plaque load locally in vivo. These findings support CAR-Ms as a platform to rationally target, resorb, and degrade pathogenic material that accumulates with age, as exemplified by targeting Aβ in AD.

### Methods

Plasmids: The Aβ CAR construct was generated by adding the aducanumab scFv, and mFcγR extracellular, transmembrane, and intracellular domains to a MuLV retroviral backbone. The control CAR constructs were generated by adding an anti-EphA2 scFv with or without the FcγR intracellular domain to the retroviral backbone. The GFP-luc construct was made from GFP and F-luciferase in a MuLV retroviral backbone. The M-CSF construct was generated by adding Thy1.1 and M-CSF to the retroviral backbone.

Cell lines: RD114 and Plat-E cells were cultured in DMEM media (Sigma-Aldrich) with 10% FBS (Atlas Biologicals), 1% penicillin and streptomycin (Gibco). The generation of HoxB8 cells has been previously described. Briefly, femoral bone marrow cells from C57BL/6J mice were isolated and cultured in recombinant mouse IL-3, IL-6, and SCF for 2 days. On the third day of culture, cells were cultured in media with FLT3-ligand and infected with retroviral introduction of an MSCV vector containing the mouse Hoxb8 DNA and a human estrogen receptor binding domain with a mutation that prevents physiological concentrations of estrogen from binding. HoxB8 cells were maintained in RPMI media (Gibco) with 10% FBS (Atlas Biologicals), 1% penicillin and streptomycin (Gibco), 1% sodium pyruvate (Gibco), non-essential amino acids (Gibco), HEPES (Thermo-Fischer), 2Mm L-glutamine, 0.1% beta-mercaptoethanol (Gibco), FLT3-ligand and beta estradiol (Sigma). All the HoxB8 cells were transduced with retroviral vectors obtained from Plat-E cell lines. After transduction, FACS sorting is performed to obtain 100% retroviral vector+ HoxB8 cells. To induce differentiation, sorted HoxB8 cells were incubated in RPMI media supplemented with 10% M-CSF for 6 days. Macrophages were lifted from cell culture plates for downstream assays with Accutase (Innovative Cell Technologies). All the cell lines were regularly tested for mycoplasma by in-house PCR.

Cell transfection and transduction: The RD114 retroviral packaging cell line was seeded to 6-well plates at a concentration of 500,000 cells/well the day before transfection. 2.5 μg of plasmid DNA was mixed with 5 μL Polyethyleneimine (PEI) at 1 ug/ml stock solution (Alfa Aesar) and incubated for 15 min at room temperature before being added to the RD114 cells. 16-24 hours later, media was changed and retrovirus was collected for use starting 24 hours later. RD114 virus was used to generate stable virus-producer Plat-E cell lines generated via retroviral transduction with 8 μg/ml polybrene and spinfection at 3000 rpm for 1 hr. Plat-E virus was used to transduce HoxB8 cells, which were transduced with 4 μg/ml polybrene and spinfection at 600×g for 30 mins.

Flow cytometry: All antibodies were titrated. CAR expression was measured with Goat anti-human IgG Alexa Fluor 647 (cat. 156339, Jackson ImmunoResearch) and transduction with the M-CSF construct was measured with anti-Thy1.1 PE (clone OX-7, cat. 202524, BioLegend). M-CSF differentiated macrophages were stained with Fc block and Oligoblock (made in-house), anti-F4/80 BV421 (clone T45-2342, cat. 565411, BD BioSciences) and anti-CD64 BV786 (clone X54-5/7.1, cat. 569507, BD BioSciences). Macrophages incubated on APP/PS1 brain slices were phenotyped by staining with anti-CD86 BV605 (clone GL-1, cat. 105037, BioLegend), anti-MHC-II FITC (clone M5/114.15.2, cat. 107606, BioLegend), anti-PD-L1 BV421 (clone MIH5, cat. 564716, BD BioSciences), anti-CD40 PeCy5 (clone 3/23, cat. 124618, BioLegend), and anti-CD206 BV711 (clone C068C2, cat. 141727, BioLegend). Live/dead staining and the M-CSF CAR-M viability assay was conducted with ZombieNIR Fixable Viabilty Dye (cat. 423106, Biolegend). M-CSF reinforced macrophages were phenotyped with additional markers: anti-F4/80 PerCP-Vio700 (clone REA126, cat.130-118-466, Miltenyi), anti-CCR2 PE (clone QA18A56, cat. 160105, Biolegend), anti-CD62L BV711 (clone MEL14, cat. 104445, Biolegend), anti-Ly6C APC(clone HK1.4, cat. 128015, Biolegend). Flow cytometry data was acquired on a Cytek NL-3000, and data was analyzed with FlowJo version 10.8.1 (Treestar/BD Biosciences).

In vitro Aβ phagocytosis assay: Untransduced or CAR-macrophages were generated by differentiating HoxB8 cells with M-CSF for 6 days and were then seeded in 96-well plates at a concentration of 20,000/well in 200 μL RPMI. 1 μL of either HiLyte™ Fluor 488-labeled Amyloid-beta (1-42) (Anaspec) prepared by reconstituting 0.1 mg in 50 μL of 1% NH₄OH or AF488 labeled, was added into each well. After 2 and 4 hours of co-incubation, the wells were washed with PBS, stained with ZombieNIR Fixable Viability dye and anti-F4/80 BV421, and analyzed by flow cytometry. Some of the cells were plated onto eight-well chamber slides (Ibidi, cat #80826) for confocal microscopy. Briefly, cells were permeabilized and blocked with 0.3% Triton X-100/3% dry milk in 0.01M PBS for 15 mins, followed by incubation with primary antibodies overnight at 4° C. and fluorescently labeled secondary antibodies at 37° C. for 1 hr (primary and secondary antibodies shown in Supplemental Table 1). After staining, cells were rinsed in PBS and images were acquired on a Nikon AXR laser scanning confocal microscope with a 60× oil immersion objective. Image analysis was conducted using Image J.

Ex vivo plaque phagocytosis assay, degradation assay, and CAR-M phenotyping: 12-14 month old APP/PS1 mice were perfused with PBS prior to brain extraction. Brains were frozen on dry ice and then stored at −80° C. Prior to each assay, frozen brains were sectioned into 10 μm slices using a CryoStat, and subsequently placed on poly-D-lysine coated coverslips. The coverslips were then placed in cell culture plates immersed in culture media.

CAR Hoxb8 cells were induced to differentiate to macrophages in RPMI media with M-CSF for 6 days. On day 6, floating cells were washed away with PBS and adherent CAR-Macrophages were lifted with Accutase (Innovative Cell Technologies). Brain slices were incubated with 2×10⁵ untransduced macrophages, control CAR-M, or Aβ CAR-Ms in 1 mL of differentiation media (RPMI+M-CSF) for 44 hours. Cells were incubated on adjacent brain slices to approximately match plaque load and distribution across conditions. For phenotyping, macrophages were lifted from the brain slices using Trypsin EDTA (0.05%, Gibco cat. 25300054) and stained for flow cytometry as above. For plaque analysis, cells were cultured on adjacent brain slices to approximately match plaque load and distribution. After incubation, brain slices were fixed in 4% PFA for 20 min. Slices were then stained with X-34 dye or HJ3.4 antibody. High resolution images of the slices were taken using a NanoZoomer Digital Scanner (Hamamatsu Photonics). The total area of plaque coverage was measured with NIH ImageJ software and expressed as percentage total area. The frequency of size classified plaques was analyzed with ImageJ software.

To analyze intracellular plaque degradation, macrophages were lifted from the brain slices using Trypsin EDTA and passaged into 8-well chamber slides (Lab-Tek cat. 154941). After incubation for 0.5 hr, 1 hr, 2 hrs and 4 hrs, cells were fixed with 4% PFA and then stained with HJ3.4 antibody and DAPI. Images were acquired using Zeiss LSM 980 Airyscan 2 confocal microscope. Surface area of intracellular plaques and cell counting were analyzed with Imaris 10.0 software and expressed as area per cell.

Intracranial injection and BLI imaging: APP/PS1 mice (B6;C3-Tg(APPswe,PSEN1dE9)85Dbo/Mmjax from Jackson Labs, MMRRC Stock No. 34928, maintained as C57BL/6× C3H strain), 12-14 month of age, were preconditioned with either 20 mg/kg Busulfan once a day for 2 days or PLX5622 (HY-114153, MedChemExpress) at 50 mg/kg twice daily for 4 days by intraperitoneal (IP) injection. Busulfan was dissolved in DMSO (Sigma) at a concentration of 30 mg/mL and diluted to 3 mg/mL in PBS for injection. PLX5622 was dissolved in DMSO at a concentration of 50 mg/mL and diluted to 5 mg/mL in in 20% Kolliphor RH40 (Sigma-Aldrich) in PBS.

Cells were prepared for intracranial injection by incubating CAR HoxB8 cells in 10% M-CSF differentiation media for 6 days. On Day 6 post differentiation, the macrophages were lifted with Accutase, washed 3 times with PBS, and loaded into a 5 μL Hamilton syringe at a concentration of 1.5×10⁵ cells/μL. 2 μL of Aβ CAR-Ms were injected into the right hippocampus and 2 μL of PBS or control CAR-Ms were injected into the left hippocampus (Coordinate: AP: −2.0, ML: ±1.6 DV: −1.5). 200 ng/ml recombinant M-CSF was added to the cell preparations to improve cell viability during injections. Bioluminescence imaging (BLI) was performed every 3-7 days; mouse heads were shaved before imaging. Mice were grouped in three-day intervals when charting BLI over time.

Immunohistochemistry and plaque staining for in vivo studies: 12-14 month old male and female APP/PS1 mice were sacrificed on day 14 with Fatal-Plus and perfused with PBS. For microglia depletion studies (FIG. 5C), mice were sacrificed on day 4 post preconditioning with PBS, Busulfan, or PLX5622 as above. Brains were removed, fixed for 24 hours in 4% paraformaldehyde fixative in 0.1 M phosphate buffer (PB) (pH 7.4) and then transferred to a solution containing 30% sucrose in 0.1 M PB until the tissue sank down. The brain was then sectioned (40 m) and twelve equally spaced sections (80 m apart) containing the dorsal part of hippocampus were immunostained using antibodies against GFP and HJ3.4 delineated below. For microglia depletion studies, brain sections were stained with Iba1. Brain sections were permeabilized and blocked with 0.3% Triton X-100/3% dry milk in 0.01 M PBS for 30 minutes followed by incubation with primary antibodies overnight at 4° C. and fluorescently labeled secondary antibodies at 37° C. for 1 hour. Primary and secondary antibodies employed are shown in Table 1. For X-34 staining, brain slices were mounted on glass slides. Tissue was permeabilized with 0.25% Triton for 30 minutes and stained with X-34 dissolved in a solution of 40% ethanol in water, pH 10, for 20 minutes. The tissue was then rinsed in distilled water and mounted.

TABLE 1
Primary and secondary antibodies used for immunohistochemistry and plaque staining

| | Primary working | | Secondary working | |
|---|---|---|---|---|
| Antigen | concentration | Source | concentration | Source |
| Lamp1 | 1:100 | Rat anti-Lamp1 | 1:800 | Donkey anti-rat Cy3 |
| | | (Santa Cruz | | (Jackson |
| | | Biotechnology, | | ImmunoResearch |
| | | cat# sc-19992) | | Inc., cat# 712-165- |
| | | | | 153) |
| Iba1 | 1:1000 | Rabbit anti-Iba1 | 1:800 | Donkey anti-rabbit |
| | | (Wako Pure | | Alexa Flour 488 |
| | | Chemical Industries, | | (Thermo Fisher) |
| | | Ltd. | | |
| | | cat# 019-19741) | | |
| GFP | 1:500 | Goat anti-GFP | 1:800 | Anti-goat Alexa Flour |
| | | (Rockland | | 488 (Life Sciences) |
| | | antibodies) | | |
| Aβ (HJ3.4) | 1:1000 | Biotinylated | 1:800 | Cy5 Streptavidin |
| | | antibody, gift | | (Jackson |
| | | from Dr. David M. | | ImmunoResearch |
| | | Holtzman | | Inc.) |

Assessment of Microglial Depletion with Preconditioning: Images were acquired with a Biotek Cytation 5 (Agilent) and images were analyzed with Biotek Gen5 software. For each mouse, six regions of interest (ROIs) were captured in each hippocampus at 20× magnification, for a total of 12 ROIs per mouse and three mice per group. In each ROI, Iba-1 positive cells were counted using the following parameters in Gen5: threshold minimum 4800, minimum size 10 am, maximum size 60 am, include primary edge objects, do not split touching objects. This mask was applied using the same settings to all ROIs across conditions to quantify microglia in the hippocampus.

Quantification of amyloid plaque load: Images were acquired using a Zeiss Axio Scan Z1 or a Nikon AXR Confocal Microscope. For each mouse, five to nine sections containing the dorsal hippocampus (spaced 80 am apart) were analyzed using Image J. Circular regions of interest (ROIs) with uniform areas of 3622 μm² were created, centered around the injection site in the hippocampus (residual GFP signal). Plaque load was quantified within these circles using brain sections stained with either HJ3.4 or X-34. Plaque load was expressed as percentage of area within the ROL. Uniform circle sizes were used across all slices. For controls, circular ROIs in mirror cortical regions were analyzed to ensure the density of plaques was relatively uniform across mirror regions of each hemisphere.

Statistics: Statistical analyses were performed in GraphPad Prism version 9.5.1. Results are expressed as mean±s.e.m. Statistical differences were assessed with the unpaired 2-tailed Student's t test for comparison of 2 experimental groups or ANOVA for 3+ experimental groups. P values from ANOVA with multiple-comparisons were generated with Tukey's multiple-comparisons test. Specific test used in each case is denoted in figure legends. Unless otherwise stated, *P<0.05, **P<0.01, ***P<0.001, ****P<0.0001. A 2-tailed P value of less than 0.05 was considered statistically significant.

Study approval: All animal studies were approved by the IACUC at Washington University School of Medicine.

TABLE 2
Sequences

| SEQ | | |
|---|---|---|
| ID | | |
| NO. | Name | Sequence |
| 1 | Aducanumab VH | QVQLVESGGGVVQPGRSLRLSCAASGFAFSSYGMH |
| | | WVRQAPGKGLEWVAVIWFDGTKKYYTDSVKGRFTI |
| | | SRDNSKNTLYLQMNTLRAEDTAVYYCARDRGIGAR |
| | | RGPYYMDVWGKGTTVTVSSASTKGPSVFPLAPSSKS |
| | | TSGGTAALGCLVKDYFPEPVTVSWNSGALTSGVHTF |
| | | PAVLQSSGLYSLSSVVTVPSSSLGTQTYICNVNHKPS |
| | | NTKVDKRVEPKSC |
| 2 | Aducanumab VL | DIQMTQSPSSLSASVGDRVTITCRASQSISSYLNWYQ |
| | | QKPGKAPKLLIYAASSLQSGVPSRFSGSGSGTDFTLTI |
| | | SSLQPEDFATYYCQQSYSTPLTFGGGTKVEIKRTVAA |
| | | PSVFIFPPSDEQLKSGTASVVCLLNNFYPREAKVQWK |
| | | VDNALQSGNSQESVTEQDSKDSTYSLSSTLTLSKAD |
| | | YEKHKVYACEVTHQGLSSPVTKSFNRGEC |
| 3 | Car Extracellular | LDEPQLCYILDAVLFLYGIVLTLLYCRLKIQVRKAAI |
| | Linker, Transmembrane | ASREKADAVYTGLNTRSQETYETLKHEKPPQ |
| | And Intracellular | |
| | Domain Derived From | |
| | The Common Gamma | |
| | Chain | |
| 4 | Car Extracellular linker | LDEPQ |
| 5 | Mouse Forg | LCYILDAVLFLYGIVLTLLYC |
| | Transmembrane | |
| 6 | Mouse Forg | RLKIQVRKAAIASREKADAVYTGLNTRSQETYETLK |
| | Intracellular domain | HEKPPQ |
| | derived from gamma | |
| | chain | |
| 7 | Human Transmembrane | LCYILDAILFLYGIVLTLLYC |
| 8 | Human Intracellular | RLKIQVRKAAITSYEKSDGVYTGLSTRNQETYETLKH |
| | domain | EKPPQ |
| 9 | VH-VL linker | GGGGSGGGGSGGGGS |
| 10 | XhoI-FLAG-mFcrg EC | LEDYKDDDDKLDEPQ |
| | (8 + 5 aa) | |
| 11 | Full CAR sequence- | QVQLVESGGGVVQPGRSLRLSCAASGFAFSSYGMH |
| | Aducanumab with | WVRQAPGKGLEWVAVIWFDGTKKYYTDSVKGRFTI |
| | murine FcRy (without | SRDNSKNTLYLQMNTLRAEDTAVYYCARDRGIGAR |
| | signal peptide) | RGPYYMDVWGKGTTVTVSSASTKGPSVFPLAPSSKS |
| | | TSGGTAALGCLVKDYFPEPVTVSWNSGALTSGVHTF |
| | | PAVLQSSGLYSLSSVVTVPSSSLGTQTYICNVNHKPS |
| | | NTKVDKRVEPKSCGGGGSGGGGSGGGGSDIQMTQS |
| | | PSSLSASVGDRVTITCRASQSISSYLNWYQQKPGKAP |
| | | KLLIYAASSLQSGVPSRFSGSGSGTDFTLTISSLQPEDF |
| | | ATYYCQQSYSTPLTFGGGTKVEIKRTVAAPSVFIFPPS |
| | | DEQLKSGTASVVCLLNNFYPREAKVQWKVDNALQS |
| | | GNSQESVTEQDSKDSTYSLSSTLTLSKADYEKHKVY |
| | | ACEVTHQGLSSPVTKSFNRGECLEDYKDDDDKLDEP |
| | | QLCYILDAVLFLYGIVLTLLYCRLKIQVRKAAIASRE |
| | | KADVYTGLNTRSQETYETLKHEKPPQ |
| 12 | Full CAR sequence- | VQLVESGGGVVQPGRSLRLSCAASGFAFSSYGMHW |
| | Aducanumab with | VRQAPGKGLEWVAVIWFDGTKKYYTDSVKGRFTIS |
| | human FcRy (without | RDNSKNTLYLQMNTLRAEDTAVYYCARDRGIGARR |
| | signal peptide) | GPYYMDVWGKGTTVTVSSASTKGPSVFPLAPSSKST |
| | | SGGTAALGCLVKDYFPEPVTVSWNSGALTSGVHTFP |
| | | AVLQSSGLYSLSSVVTVPSSSLGTQTYICNVNHKPSN |
| | | TKVDKRVEPKSCGGGGSGGGGSGGGGSDIQMTQSPS |
| | | SLSASVGDRVTITCRASQSISSYLNWYQQKPGKAPKL |
| | | LIYAASSLQSGVPSRFSGSGSGTDFTLTISSLQPEDFAT |
| | | YYCQQSYSTPLTFGGGTKVEIKRTVAAPSVFIFPPSDE |
| | | QLKSGTASVVCLLNNFYPREAKVQWKVDNALQSGN |
| | | SQESVTEQDSKDSTYSLSSTLTLSKADYEKHKVYAC |
| | | EVTHQGLSSPVTKSFNRGECLEDYKDDDDKLDEPQL |
| | | CYILDAILFLYGIVLTLLYCRLKIQVRKAAITSYEKSD |
| | | GVYTGLSTRNQETYETLKHEKPPQ |
| 13 | Secreted MCSF Mouse | MTAPGAAGRCPPTTWLGSLLLLVCLLASRSITEEVSE |
| | | YCSHMIGSGHLQSLQRLIDSQMETSCQITFEFVDQEQ |
| | | LKDPVCYLKKAFLLVQDIMEDTMRFRDNTPNAIAIV |
| | | QLQELSLRLKSCFTKDYEEHDKACVRTFYETPLQLLE |
| | | KVKNVFNETKNLLDKDWNIFSKNCNNSFAECSSQDV |
| | | VTKPDCN |
| 14 | Secreted MCSF human | MTAPGAAGRCPPTTWLGSLLLLVCLLASRSITEEVSE |
| | | YCSHMIGSGHLQSLQRLIDSQMETSCQITFEFVDQEQ |
| | | LKDPVCYLKKAFLLVQDIMEDTMRFRDNTPNAIAIV |
| | | QLQELSLRLKSCFTKDYEEHDKACVRTFYETPLQLLE |
| | | KVKNVFNETKNLLDKDWNIFSKNCNNSFAECSSQDV |
| | | VTKPDCN |
| 15 | Donenemb VH | QVQLVQSGAEVKKPGSSVKVSCKASGYDFTRYYIN |
| | | WVRQAPGQGLEWMGWINPGSGNTKYNEKFKGRVTI |
| | | TADESTSTAYMELSSLRSEDTAVYYCAREGITVYWG |
| | | QGTTVTVSSASTKGPSVFPLAPSSKSTSGGTAALGCL |
| | | VKDYFPEPVTVSWNSGALTSGVHTFPAVLQSSGLYS |
| | | LSSVVTVPSSSLGTQTYICNVNHKPSNTKVDKKVEPK |
| | | SCDKTHTCPPCPAPELLGGPSVFLFPPKPKDTLMISRT |
| | | PEVTCVVVDVSHEDPEVKFNWYVDGVEVHNAKTKP |
| | | REEQYNSTYRVVSVLTVLHQDWLNGKEYKCKVSNK |
| | | ALPAPIEKTISKAKGQPREPQVYTLPPSRDELTKNQVS |
| | | LTCLVKGFYPSDIAVEWESNGQPENNYKTTPPVLDS |
| | | DGSFFLYSKLTVDKSRWQQGNVFSCSVMHEALHNH |
| | | YTQKSLSLSPG |
| 16 | Donenemb VL | DIVMTQTPLSLSVTPGQPASISCKSSQSLLYSRGKTYL |
| | | NWLLQKPGQSPQLLIYAVSKLDSGVPDRFSGSGSGT |
| | | DFTLKISRVEAEDVGVYYCVQGTHYPFTFGQGTKLEI |
| | | KRTVAAPSVFIFPPSDEQLKSGTASVVCLLNNFYPRE |
| | | AKVQWKVDNALQSGNSQESVTEQDSKDSTYSLSSTL |
| | | TLSKADYEKHKVYACEVTHQGLSSPVTKSFNRGEC |
| 17 | Human GMCSF | APARSPSPSTQPWEHVNAIQEARRLLNLSRDTAAEM |
| | | NETVEVISEMFDLQEPTCLQTRLELYKQGLRGSLTKL |
| | | KGPLTMMASHYKQHCPPTPETSCATQTITFESFKENL |
| | | KDFLLVIPFDCWEPVQE |
| 18 | Aducanumab VH- | QVQLVESGGGVVQPGRSLRLSCAASGFAFSSYGMH |
| | linker-Aducanumab | WVRQAPGKGLEWVAVIWFDGTKKYYTDSVKGRFTI |
| | VL | SRDNSKNTLYLQMNTLRAEDTAVYYCARDRGIGAR |
| | | RGPYYMDVWGKGTTVTVSSASTKGPSVFPLAPSSKS |
| | | TSGGTAALGCLVKDYFPEPVTVSWNSGALTSGVHTF |
| | | PAVLQSSGLYSLSSVVTVPSSSLGTQTYICNVNHKPS |
| | | NTKVDKRVEPKSCGGGGSGGGGSGGGGSDIQMTQSP |
| | | SSLSASVGDRVTITCRASQSISSYLNWYQQKPGKAP |
| | | KLLIYAASSLQSGVPSRFSGSGSGTDFTLTISSLQP |
| | | EDFATYYCQQSYSTPLTFGGGTKVEIKRTVAAPSVF |
| | | IFPPSDEQLKSGTASVVCLLNNFYPREAKVQWKVDN |
| | | ALQSGNSQESVTEQDSKDSTYSLSSTLTLSKADYEK |
| | | HKVYACEVTHQGLSSPVTKSFNRGEC |
| 19 | Donenemb VH-linker- | QVQLVQSGAEVKKPGSSVKVSCKASGYDFTRYYIN |
| | Donenemb VL | WVRQAPGQGLEWMGWINPGSGNTKYNEKFKGRVTI |
| | | TADESTSTAYMELSSLRSEDTAVYYCAREGITVYW |
| | | GQGTTVTVSSASTKGPSVFPLAPSSKSTSGGTAAL |
| | | GCLVKDYFPEPVTVSWNSGALTSGVHTFPAVLQSS |
| | | GLYSLSSVVTVPSSSLGTQTYICNVNHKPSNTKVD |
| | | KKVEPKSCDKTHTCPPCPAPELLGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALPAPIEKTISKAKGQPREPQVYTLP |
| | | PSRDELTKNQVSLTCLVKGFYPSDIAVEWESNGQP |
| | | ENNYKTTPPVLDSDGSFFLYSKLTVDKSRWQQGNV |
| | | FSCSVMHEALHNHYTQKSLSLSPGGGGGSGGGGSG |
| | | GGGSDIVMTQTPLSLSVTPGQPASISCKSSQSLLY |
| | | SRGKTYLNWLLQKPGQSPQLLIYAVSKLDSGVPDR |
| | | FSGSGSGTDFTLKISRVEAEDVGVYYCVQGTHYPF |
| | | TFGQGTKLEIKRTVAAPSVFIFPPSDEQLKSGTAS |
| | | VVCLLNNFYPREAKVQWKVDNALQSGNSQESVTEQ |
| | | DSKDSTYSLSSTLTLSKADYEKHKVYACEVTHQGL |
| | | SSPVTKSFNRGEC |
| 20 | Signal peptide | MEFGLSWVFLVALFRGVQC |
| 21 | Full CAR sequence- | QVQLVQSGAEVKKPGSSVKVSCKASGYDFTRYYIN |
| | Donenemb with murine | WVRQAPGQGLEWMGWINPGSGNTKYNEKFKGRVTI |
| | FcRγ (without signal | TADESTSTAYMELSSLRSEDTAVYYCAREGITVYWG |
| | peptide) | QGTTVTVSSASTKGPSVFPLAPSSKSTSGGTAALGCL |
| | | VKDYFPEPVTVSWNSGALTSGVHTFPAVLQSSGLYS |
| | | LSSVVTVPSSSLGTQTYICNVNHKPSNTKVDKKVEPK |
| | | SCDKTHTCPPCPAPELLGGPSVFLFPPKPKDTLMISRT |
| | | PEVTCVVVDVSHEDPEVKFNWYVDGVEVHNAKTKP |
| | | REEQYNSTYRVVSVLTVLHQDWLNGKEYKCKVSNK |
| | | ALPAPIEKTISKAKGQPREPQVYTLPPSRDELTKNQVS |
| | | LTCLVKGFYPSDIAVEWESNGQPENNYKTTPPVLDS |
| | | DGSFFLYSKLTVDKSRWQQGNVFSCSVMHEALHNH |
| | | YTQKSLSLSPGGGGGSGGGGSGGGGSDIVMTQTPLS |
| | | LSVTPGQPASISCKSSQSLLYSRGKTYLNWLLQKPGQ |
| | | SPQLLIYAVSKLDSGVPDRFSGSGSGTDFTLKISRVEA |
| | | EDVGVYYCVQGTHYPFTFGQGTKLEIKRTVAAPSVFI |
| | | FPPSDEQLKSGTASVVCLLNNFYPREAKVQWKVDN |
| | | ALQSGNSQESVTEQDSKDSTYSLSSTLTLSKADYEKH |
| | | KVYACEVTHQGLSSPVTKSFNRGECLEDYKDDDDK |
| | | LDEPQLCYILDAVLFLYGIVLTLLYCRLKIQVRKAAI |
| | | ASREKADAVYTGLNTRSQETYETLKHEKPPQ |
| 22 | Full CAR sequence- | QVQLVQSGAEVKKPGSSVKVSCKASGYDFTRYYIN |
| | Donenemb with human | WVRQAPGQGLEWMGWINPGSGNTKYNEKFKGRVTI |
| | FcRγ (without signal | TADESTSTAYMELSSLRSEDTAVYYCAREGITVYWG |
| | peptide) | QGTTVTVSSASTKGPSVFPLAPSSKSTSGGTAALGCL |
| | | VKDYFPEPVTVSWNSGALTSGVHTFPAVLQSSGLYS |
| | | LSSVVTVPSSSLGTQTYICNVNHKPSNTKVDKKVEPK |
| | | SCDKTHTCPPCPAPELLGGPSVFLFPPKPKDTLMISRT |
| | | PEVTCVVVDVSHEDPEVKFNWYVDGVEVHNAKTKP |
| | | REEQYNSTYRVVSVLTVLHQDWLNGKEYKCKVSNK |
| | | ALPAPIEKTISKAKGQPREPQVYTLPPSRDELTKNQVS |
| | | LTCLVKGFYPSDIAVEWESNGQPENNYKTTPPVLDS |
| | | DGSFFLYSKLTVDKSRWQQGNVFSCSVMHEALHNH |
| | | YTQKSLSLSPG |
| | | GGGGSGGGGSGGGGSDIVMTQTPLSLSVTPGQPASIS |
| | | CKSSQSLLYSRGKTYLNWLLQKPGQSPQLLIYAVSK |
| | | LDSGVPDRFSGSGSGTDFTLKISRVEAEDVGVYYCV |
| | | QGTHYPFTFGQGTKLEIKRTVAAPSVFIFPPSDEQLKS |
| | | GTASVVCLLNNFYPREAKVQWKVDNALQSGNSQES |
| | | VTEQDSKDSTYSLSSTLTLSKADYEKHKVYACEVTH |
| | | QGLSSPVTKSFNRGECLEDYKDDDDKLDEPQLCYIL |
| | | DAILFLYGIVLTLLYCRLKIQVRKAAITSYEKSDGVY |
| | | TGLSTRNQETYETLKHEKPPQ |

What is claimed is:

1. A chimeric antigen receptor (CAR) comprising an antigen binding domain that specifically binds to beta amyloid (Aβ).

2. The CAR of claim 1, wherein the Aβ is an aggregated form of Aβ.

3. The CAR of claim 1, wherein the antigen binding domain comprises an anti-Aβ antibody or an antigen binding fragment thereof.

4. The CAR of claim 1, wherein the antigen binding domain comprises an anti-Aβ single chain variable fragment (scFv).

5. The CAR of claim 4, wherein the antigen binding domain comprises: (a) a heavy chain variable region comprising the amino acid sequence set forth in SEQ ID NO: 1, and a light chain variable region comprising the amino acid sequence set forth in SEQ ID NO: 2; or (b) a heavy chain variable region comprising the amino acid sequence set forth in SEQ ID NO: 15, and a light chain variable region comprising the amino acid sequence set forth in SEQ ID NO: 16.

6. The CAR of claim 1, further comprising a linker, a transmembrane domain, and an intracellular domain.

7. The CAR of claim 6, wherein the linker comprises the amino acid sequence set forth in SEQ ID NO: 4.

8. The CAR of claim 6, wherein the transmembrane domain comprises the amino acid sequence set forth in SEQ ID NO: 5 or SEQ ID NO: 7.

9. The CAR of claim 6, wherein the intracellular domain comprises a FcRγ signaling domain.

10. The CAR of claim 9, wherein the FcRγ signaling domain comprises the amino acid sequence set forth in SEQ ID NO: 6 or SEQ ID NO: 8.

11. A vector comprising a nucleic acid encoding the CAR of claim 1.

12. The vector of claim 11, wherein the vector is a retrovirus or a lentivirus.

13. The vector of claim 12, wherein the retrovirus is Moloney Murine Leukemia Virus (MuLV).

14. A cell expressing the CAR of claim 1.

15. The cell of claim 14, wherein the cell is a macrophage.

16. The cell of claim 14, wherein the cell further expresses a cytokine.

17. The cell of claim 16, wherein the cytokine is macrophage colony-stimulating factor (M-CSF) or Granulocyte-macrophage colony-stimulating factor (GM-CSF).

18. The cell of claim 17, wherein (a) the cytokine is M-CSF and comprises the amino acid sequence set forth in SEQ ID NO: 13 or SEQ ID NO: 14; or (b) the cytokine is GM-CSF and comprises the amino acid sequence set forth in SEQ ID NO: 17.

19. The cell of claim 16, wherein the cytokine is encoded by (a) the same vector encoding the CAR; or (b) a second vector.

20. A composition comprising the cell of claim 14 and a pharmaceutically acceptable excipient.

21. A method of reducing Aβ plaques in a subject in need thereof, comprising administering to the subject the cell of claim 14.

22. The method of claim 21, wherein the cell is administered by intracranial injection.

23. The method of claim 21, wherein the method induces resorption and/or degradation of Aβ plaques.

24. The method of claim 21, wherein the cell further expresses a cytokine.

25. The method of claim 24, wherein the cytokine is M-CSF or GM-CSF.

26. A method of treating Alzheimer's disease in a subject in need thereof, comprising administering to the subject the cell of claim 14.

27. The method of claim 26, wherein the cell further expresses a cytokine.

28. The method of claim 27, wherein the cytokine is M-CSF or GM-CSF.

29. A method of reducing one or more symptoms associated with Alzheimer's disease in a subject in need thereof, comprising administering to the subject the cell of claim 14.

30. The method of claim 29, wherein the symptoms comprise diffuse or compact Aβ plaques in the subject.

31. The method of claim 29, wherein the cell further expresses a cytokine.

32. The method of claim 31, wherein the cytokine is M-CSF or GM-CSF.

33. A chimeric antigen receptor macrophage (CAR-M) comprising the amino acid sequence set forth in any one of SEQ ID NO: 11, SEQ ID NO: 12, SEQ ID NO: 20, or SEQ ID NO: 22.


SEQUENCE LISTING (22 sequences)

SEQ ID NO: 1 - protein, 227 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 2 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 68 aa, synthetic construct
LDEPQLCYILDAVLFLYGIVLTLLYCRLKIQVRKAAIASREKADAVYTGLNTRSQETYETLKHEKPPQ

SEQ ID NO: 4 - protein, 5 aa, synthetic construct
LDEPQ

SEQ ID NO: 5 - protein, 21 aa, Mus musculus
LCYILDAVLFLYGIVLTLLYC

SEQ ID NO: 6 - protein, 42 aa, Mus musculus
RLKIQVRKAAIASREKADAVYTGLNTRSQETYETLKHEKPPQ

SEQ ID NO: 7 - protein, 21 aa, Homo sapiens
LCYILDAILFLYGIVLTLLYC

SEQ ID NO: 8 - protein, 42 aa, Homo sapiens
RLKIQVRKAAITSYEKSDGVYTGLSTRNQETYETLKHEKPPQ

SEQ ID NO: 9 - protein, 15 aa, synthetic construct
GGGGSGGGGSGGGGS

SEQ ID NO: 10 - protein, 15 aa, synthetic construct
LEDYKDDDDKLDEPQ

SEQ ID NO: 11 - protein, 533 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEDYKDDDDKLDEPQLCYILDAVLFLYGIVLTLLYCRLKIQVRKAAIASREKADVYTGLNTRSQETYETLKHEKPPQ

SEQ ID NO: 12 - protein, 533 aa, synthetic construct
VQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEDYKDDDDKLDEPQLCYILDAILFLYGIVLTLLYCRLKIQVRKAAITSYEKSDGVYTGLSTRNQETYETLKHEKPPQ

SEQ ID NO: 13 - protein, 190 aa, Mus musculus
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCN

SEQ ID NO: 14 - protein, 190 aa, Homo sapiens; identical to SEQ ID NO: 13

SEQ ID NO: 15 - protein, 444 aa, synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 219 aa, synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 127 aa, Homo sapiens
APARSPSPSTQPWEHVNAIQEARRLLNLSRDTAAEMNETVEVISEMFDLQEPTCLQTRLELYKQGLRGSLTKLKGPLTMMASHYKQHCPPTPETSCATQTITFESFKENLKDFLLVIPFDCWEPVQE

SEQ ID NO: 18 - protein, 456 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 678 aa, synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 19 aa, synthetic construct
MEFGLSWVFLVALFRGVQC

SEQ ID NO: 21 - protein, 756 aa, synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEDYKDDDDKLDEPQLCYILDAVLFLYGIVLTLLYCRLKIQVRKAAIASREKADAVYTGLNTRSQETYETLKHEKPPQ

SEQ ID NO: 22 - protein, 756 aa, synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSDIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLEDYKDDDDKLDEPQLCYILDAILFLYGIVLTLLYCRLKIQVRKAAITSYEKSDGVYTGLSTRNQETYETLKHEKPPQ